(19) United States
(12) Patent Application Publication
(10) Pub. No.: US 2021/0002595 A1
(43) Pub. Date: Jan. 7, 2021

(54) CULTURE TANK

(71) Applicant: Saganatura EHF., Hafnarfjordur (IS)

(72) Inventors: Sjofn SIGURGUSLADOTTIR, Kopavogur (IS); Tryggvi E. MATHIESEN, Reykjavik (IS); Sigurbjorn EINARSSON, Reykjavik (IS)

(21) Appl. No.: 16/769,347

(22) PCT Filed: Mar. 11, 2019
(86) PCT No.: PCT/IS2019/050002

§ 371 Date: Jun. 3, 2020

(30) Foreign Application Priority Data
Mar. 12, 2018 (IS) 050214

(51) Int. Cl. C12M 1/00; C12M 1/02; C12M 1/34

(52) U.S. Cl. CPC C12M 21/02; C12M 31/10; C12M 29/08; C12M 41/06; C12M 41/26; C12M 41/32; C12M 41/18

(57) ABSTRACT

A photobioreactor for culturing light-sensitive microbes is provided, the reactor comprising a body comprising at least one floor panel and at least one wall extending upwardly around the periphery of the floor panel to define a body, wherein the at least one floor panel comprises at least one graded segment so as to define a trough along the bottom of the body towards which debris within the body flows, at least one illumination panel within the body so that there is free flow of liquid along the sides and floor panel of the body, at least one gas inlet within the trough in the floor panel, for providing an upward stream of gas into the body so as to generate air lift of the light sensitive microbes within the body. Also provided is a method for growing light-sensitive microbes.

### FIELD

The invention is within the field of cultivation, and specifically the cultivation of light sensitive microbes, using culture tanks that comprise illumination for sustaining growth of such microbes.

### INTRODUCTION

Photosynthetic cells are of great importance as primary producers of various organic matters and for their ability to regenerate atmosphere. Utilization of biotechnological applications of the photosynthetic machinery of microbes in the production of bio-active compounds and in environmental processes has received increased attention over the last several decades. Microalgae in particular have vast potential as sources for valuable pharmaceuticals, pigments and other fine chemicals. The red ketocarotenoid pigment, astaxanthin (3,3′-dihydroxy-β,β-carotene-4,4′-dione), is a strong antioxidant and has received an increased interest from the cosmetics, the food and the feed industries. The strong antioxidant properties of astaxanthin are believed to underpin its potential therapeutic activity.

Astaxanthin is ubiquitous in nature, especially in the marine environment, and is probably best-known for eliciting the pinkish-red hue to the flesh of salmonids as well as shrimp, lobster and crayfish. Haematococcus pluvialis is considered as the most promising producer of astaxanthin, as it accumulates the (3S,3′S)-isomer of astaxanthin, mostly in its mono- and di-ester forms in cytoplasmic lipid bodies as a secondary carotenoid at an accumulation of more than 3% by weight.

Microalgae are highly productive, and are presently responsible for 50% of photosynthesis in the world. Microalgae are a rich and economically viable source of valuable natural bio-active compounds, and can be used whole as a nutrient supply; their productivity is such that from a 2 m³ of high-quality culture one can harvest enough to sustain one person. High efficiency photobioreactors are thus expected to enable secure and sufficient future supplies of safe, healthy and high-quality food and other bio-based products, thus exploiting microalgae to their full potential.

Large-scale cultivation, scale-up techniques, and commercial production of astaxanthin from H. pluvialis has received increased attention over the last years. A number of factors are believed to affect astaxanthin production and accumulation in H. pluvialis, including nutrient limitation or supplement, oxygen stress, and light intensity. Certain stress factors such as increased salinity, elevated temperature, high light intensity and deprivation of nitrate or phosphate have been used to promote accumulation of asthaxanthin. An additional variable is the type and configuration of the photobioreactor used for the growth. Thus, a properly designed photobioreactor and optimized illumination by effective light sources within the reactor are essential components of efficient growth.

A favourable quality and function of photobioreactors (PBR) for mass cultivation of microalgae is achieved through optimized compromise of several technical key features that relates to productivity, controllability and running safety.

The value of the targeted product decides the type of the technical solution to be selected for the microalgae cultivation, including biofuel, feed, food and nutraceuticals.

The economic outcome of a selected upstream technology for a given product is a complex composition of many factors.

Yet areal productivity and the predictability of the process and product quality are key features for the economics of commercial microalgae production.

Extensive production methods based on low investment facility extended over large area such as raceway ponds is regarded as suitable for low value products. For high value products such as food and neutraceuticals, intensive production methods are appropriate.

The most common design of photobioreactor are raceway ponds, tubular structures of glass or plastic in serpentine configuration and flat panel structures.

Tubular and flat panel are the most commonly used photobioreactor designs used for intensive microalgae cultivation.

Raceway ponds is a low investment production utility performing poorly in areal productivity (approx. 20 t/ha/yr; see Roger et al., Algal Research 4, 76-88 (2014)). However, the technology is applied alone or in combination with other technology for commercial production of food supplement such as phycocyanin (http://earthrise.com/about/eco-friendly-farm/) and astaxanthin (https://www.algatech.com/https://www.cyanotech.com(company/facility.html)

Several astaxanthin producers apply vertical tubular serpentine or manifold photobioreactors for their algae biomass outdoor and indoor upstream process (https://www.algatech.com/; http://bggworld.com/). (https://www.algalif.com/). High surface to volume ratio (65-80 m²m⁻³; see http://www.schott.com/tubing/english/special glass/industry environment/pbr.html) is the major benefit of tubular design, characterized by tubes made of glass or plastic, commonly 40-60 mm in diameter. The small tube diameter retains a short light path, allows for high proportion of photosynthetic active volume and thus maximizing photosynthetic efficiency.

By contrast, the density of tubes per unit ground area is rather low and similarly the illuminated culture surface per ground footprint (i.e. the surface of culture exposed per unit area of ground) is low, approximately 5.5-6.0 m²/square meter ground area. When running an indoor culturing process using such culturing system along with artificial illumination the investment in floor space becomes a challenge for the economy of the operation. Thus, when running a culturing process where the use of ground floor space is a cost contributing factor, along with artificial illumination, tubular photobioreactors are not an obvious choice for indoor upstream process of microalgae biomass.

The tubular photobioreactor is a highly fragmented structure, consisting of many carrying elements and glass tubes terminally coupled to each other creating grooves in the connections. These numerous grooves are places where biomass can accumulate and hide making cleaning of the facility challenging.

Flat-panel photobioreactors have received increased attention in recent years and few types are currently produced and operated commercially (https://subitec.com/de; http://www.femonline.it/; www.algamo.cz). The mixing of the culture within the FPAP is typically executed through airlift by injection of air through air-sparges in the bottom of the vessel and illuminated by an externally located light source. Similar to tall tubular photobioreactors, the advantage of the FPAP is a high photosynthetic efficiency as the thickness of the plate is normally small, typically 4-5 cm. Although high volumetric productivity is achievable (0.3-0.6 g L⁻¹ d⁻¹) using flat-panel photobioreactors, still the areal output is limited (36-66 t ha⁻¹ y⁻¹)¹ due to large areal foot print of the design.

Tanks for growing microbes such as H. pluvialis are thus known in the art. For example, Choi et al. (J Microbiol Biotechnol 2011, 21:1081-1087) disclose an annular photobioreactor with internal illumination. The tank includes a sparger that is immersed in the tank, close to its bottom and fluorescent light tubes, encapsuled by a draft tube and arranged vertically in the tank. Yoo et al. (Bioprocess Biosyst Eng 2012, 35:309-315) disclose an optimized photobioreactor for culturing of H. pluvialis, the reactor being elongate with a V-shaped bottom and an internal sparger arranged close to the bottom of the reactor. The reactor is of a bag-type with external illumination.

### SUMMARY

The present inventors have discovered that photobioreactors employing an inverted layout of the flat-panel principle are especially useful for growing light-sensitive microbes. The invention relates to such photobioreactors, including their particular structural features and the arrangement of light sources and gas inlets in the photobioreactors that is particularly suitable for maintaining growth of light sensitive microbes. The invention further relates to the use of such photobioreactors in culturing light-sensitive microbes.

The invention in an aspect relates to photobioreactors employing an inverted layout of the flat-panel principle, wherein the light source is positioned within panels, preferably thin, flat panels, that are submerged into and surrounded by a culturing liquid receiving photosynthetically active irradiation from the panels for driving culture growth. The culture can be enclosed by a rounded or generally U-shaped reactor tank structure wherein the culture is mixed by air injected through sparges located in a bottom panel of the tank.

The invention provides in another aspect a photobioreactor, in particular for culturing light sensitive microbes, the photobioreactor comprising
a. a body comprising at least one floor panel and at least one wall extending upwardly around the periphery of the at least one floor panel to define a body for receiving and holding liquids; wherein the at least one floor panel comprises at least one graded segment so as to define a trough along the bottom of the body towards which debris within the body flows;
b. at least one illumination panel, the at least one illumination panel comprising illumination means adapted to emit light that is suitable for sustaining growth of the light sensitive microbes within the photobioreactor,
wherein the at least illumination panel is disposed within the body so that there is free flow of fluid along the sides and floor panel of the body;
and
c. at least one gas inlet disposed within the trough in the at least one floor panel, for providing an upward stream of gas into the body so as to generate air lift of the light sensitive microbes within the body;
   so that, during incubation of a liquid culture of microbes in the photobioreactor, microbe circulation and growth in the photobioreactor is stimulated through concomitant exposure to light emitted by the at least one illumination panel and circulation of microbes within the body that is facilitated by an upward air lift generated by the stream of gas into the body.

The invention further provides a method for growing light-sensitive microbes. In an aspect, the method comprises steps of:
a. providing a photobioreactor, the photobioreactor comprising
   i. a body comprising a floor panel and at least one wall extending upwardly around the periphery of the floor panel to define a body for receiving and holding an aqueous solution comprising microbes; wherein the floor panel comprises at least one graded segment so as to define a trough along the bottom of the body towards which the microbes can flow;
   ii. at least one illumination panel that is disposed within the body, the illumination panel comprising illumination means adapted to emit light that is suitable for sustaining growth of the light sensitive microbes within the body,
   wherein the at least one illumination panel is disposed within the body so that there is free flow of water along the at least one wall and/or floor panel; and
   iii. at least one gas inlet disposed in the trough in the floor panel, for providing an upward stream of gas in the body so as to generate air lift of the light sensitive microbes within the body;
b. providing an aqueous culture of light-sensitive microbes into the body so that the illumination panels are at least partially submerged;
c. delivering gas into the aqueous culture in the body through the at least one gas inlet, thereby generating air-lift in the body that provides an upwardly flow of microbes from the at least one gas inlet and a downwardly gravitational flow of microbes at least along and down the at least one side wall, towards the trough in the floor panel,
d. providing illumination into the body by means of the at least one illumination panel;
   whereby microbe growth is stimulated through the airlift-driven circulation of the microbes within the body and exposure to light emitted by the at least one illumination panel.

The microbes can in general be any light-sensitive microbe. In some embodiments, the microbe is selected from light-sensitive algae. In some embodiments, the microbe is selected from the group consisting of Arthrospira platensis, Chlorella vulgaris, Nannochloropsis oculata and Haematococcus pluvialis. In one embodiment, the microbe is Haematococcus pluvialis.

In the present context, the term “trough” should be understood to mean a depression within a surface, towards and along which fluid on the surface can flow. A trough can for example be represented by a long and shallow channel or depression on a surface. A trough can be in the form of a channel or conduit on a surface, a result of a groove or depression along the surface. In the context of a surface with continuous slope, a trough can be the lowermost portion of the surface, towards and along which fluid can flow.

The term “graded”, in the present context, should be understood to mean “sloping” or “having an incline”. Thus, floor panels in accordance with the invention can have at least one graded segment. This should be understood to mean that the floor panels can have at least one sloping segment, i.e. at least one segment that is at an incline (with respect to ground).

The term “air-lift”, or “airlift” in the present context, should be understood to mean the forced flow of a fluid by means of a gas into a tank, body or reactor containing a liquid. The air used for generating the air lift can be ambient air, or it can comprise, or consist of, other suitable gas. For example, the air lift can be generated by ambient air that is supplemented by other gases, such as additional oxygen gas, nitrogen gas or carbon dioxide gas.

The shape of the body can suitably be of a generally rectangular form with a rounded floor, so that a groove or trough is formed along the floor. For example, the floor can be of a generally semiannular structure. The detailed form of the floor (or bottom) can however be adjusted as needed, to influence flow characteristics and/or fluid dynamics in the tank. The floor panel and side walls of the body are preferably connected so as to form one continuous structure.

In some embodiments, the trough in the floor panel is provided by an elongated channel or depression within and along the floor panel. The photobioreactor tank has preferably continuously sloping side walls and floor panels. As a result, the trough when provided as a channel or conduit in the floor panel, will result in flow of culture in the photobioreactor tank towards and into the trough. The trough can thus be provided as the lowermost portion of the floor panel surface, towards and along which fluid can flow. This way, circulation of culture within the tank can be provided from the trough in the tank towards the upper surface of the tank, minimizing or eliminating dead spaces in the tank from which there is little or no recirculation.

The photobioreactor contains a body that is in the shape of a photobioreactor tank for holding liquids. It will thus be understood that in the following description, the terms “body”, and “tank”, “photobioreactor tank” or “reactor tank” have the same general meaning.

The side walls of the body/tank may be upright and generally planar. The side walls may also have bent or curved portions. For example, the lower part of the side walls may be curved. In an embodiment, the lower part of the side walls may be curved so as to have a curvature that is identical, or close to identical, to the curvature of a floor panel that meets the side walls.

The floor panel may comprise, or consist of, two or more panel units that each is approximately flat, and wherein the panel units meet at an angle so as to generate an overall floor panel structure that permits water and/or debris to flow towards a trough in the floor panel. The trough in the floor panel can in such embodiments, comprise a segment that consists of a flat panel unit that is approximately horizontal and is connected to at least adjacent panel units at an angle, towards side walls in the tank.

The floor panel may also be generally “V” shaped, or contain a portion that is “V” shaped. In this context, the term “V shaped” should be understood to mean two panels (preferably flat panels) that meet at an angle so as to form a structure that forms the letter “V”. It follows that such an angle between the panels is, in general, greater than 90° and less than 180°. Preferably however, such an angle is in the range of about 90° to about 150°, and preferably in the range of about 100° to about 150°.

The size and volume of the photobioreactor body is flexible and can be adapted to the number and practical size of light sources that are to be installed and the fluid dynamics properties that are required for the particular culturing.

The photobioreactor may be partially or completely double-walled, so that there is a closed volume or thermal jacket surrounding at least a portion of the inner volume of its body, for thermal regulation of culture in the photobioreactor tank (body) by circulation of temperature-controlled liquid through the thermal jacket. The thermal jacket is preferably in thermal connection with the photobioreactor tank, so that heat can be transmitted between the two thermal reservoirs, i.e. the tank and the thermal jacket. For example, the floor panel and/or the side walls may be at least partially double-walled. In some embodiments, the floor panel is double-walled. In some other embodiments, the floor panel and at least a portion of the side walls are double-walled. In such embodiments, the photobioreactor will have at least one thermal jacket inlet, for delivering water into the thermal jacket, and at least one thermal jacket outlet, for flow of thermal fluid (such as water) from the thermal jacket.

The thermal jacket preferably contains at least one continuous fluid channel, so that a fluid can be circulated within the thermal jacket. There can one or more thermal fluid controller that is adapted to control the flow rate and/or temperature of the fluid in the thermal jacket. Such controller can receive information about the temperature of culture in the photobioreactor, for example from a temperature probe in the reactor, and adjust the temperature and/or flow rate of fluid in the thermal jacket so as to maintain the culture at a preset temperature, or to change the temperature of a culture in the photobioreactor to a new temperature based on a new temperature setting. The thermal jacket can be provided as a single unit that is arranged on the outer surface of a portion of, or all of the wall and/or bottom of the photobioreactor tank. Alternatively, the thermal jacket can be provided by two or more thermal jacket units that are each provided on the outer surface of a portion of the wall and/or bottom of the photobioreactor tank.

The thermal jacket thus serves the purpose of aiding in temperature control of culture in the photobioreactor tank. There will be a circulation of a fluid, such as water, within the thermal jacket that has a temperature that is controlled to maintain culture within the tank at an appropriate and constant temperature. The thermal jacket can, during operation, be adapted to circulate fluid (e.g., water) that is at a higher or lower temperature than the temperature of the culture in the tank. Thus, if it desired to warm the circulating culture in the tank, the temperature of the thermal jacket can be raised. Alternatively, if it is desired to cool the circulating culture in the tank and/or to maintain the culture at a fixed temperature, the temperature of liquied within the thermal jacket can be lower than the temperature of the circulating culture in the tank.

There can be one or more pumps arranged on, or in fluid communication with, fluid inlets into the thermal jacket, for circulating fluid in the jacket.

In an embodiment, sea water is circulated in the thermal jacket. This can be especially important for operation of the photobioreactor in warm climates and/or in locations where natural cooling water is sparse.

In general, there can be controllers arranged for manual and/or automatic adjustment and control of growth parameters in the photobioreactor, including but not limited to temperature, pH, light (illumination), nutrients such as sources of carbon, nitrogen, phosphorous and/or silicon including but not limited to ammonia, nitrate, nitrite, orthophosphate, dissolved silica, dissolved CO₂.

Such controllers, which can be arranged as a single central controller or a collection of individual controllers, receive information about measurements of the relevant parameter (such as determination of temperature, pH, light, nutrient levels, concentration of dissolved CO₂, etc.) and in response to such measurements based on a comparison to set levels of the desirable parameter value, and send a signal to an automated valve or flow controller so as to respond to a potential deviation of measurements from the set level of the parameter.

For example, the controllers can be adapted to control the delivery of nutrients into the tank, by receiving continuous or semi-continuous information about the concentration of one or more nutrient in the tank, determine the need for supplementing one or more nutrients based on such measurements, and sending instructions to one or more pumping device and/or electronically controlled valve so as to provide increased flow of any deficient nutrients into the tank. In the same fashion, CO₂ levels in the tank, in particular levels of dissolved CO₂, can be controlled. Through control of CO₂ levels in the photobioreactor tank, the pH of the tank can be adjusted as needed; to lower the pH the rate of CO₂ injection into the tank can be increased, resulting in an increase in dissolved CO₂ which results in a decrease in pH. In other words, the CO₂ control serves as a pH stat (pH control) in the photobioreactor.

There can further more be one or more light sensors in the tank, for example in, or mounted on, the light panels in the tank. These light sensors can provide measurements of light density in the tank. One or more controllers receiving such measurements can increase and/or decrease the illumination in the tank by comparing the measured light intensity to the desired light intensity for the particular culture in the tank, and increasing or decreasing the illumination in the tank as needed.

Accordingly, the photobioreactor can be provided with at least one of the following: one or more pH meter, one or more CO₂ inlet, one or more temperature probe, one or more chemical probe and one or more optical density meter.

Alternatively or additionally, there can be channels or tubes incorporated in the photobioreactor, or surround the photobioreactor, to accommodate liquid flow for thermal regulation of culture within the photobioreactor tank.

The body or tank of the photobioreactor is preferably made from stainless steel to facilitate cleaning and disinfection and ensure thermal transport. However, it will be appreciated that the photobioreactor design is not dependent on the choice of material and the body can therefore be provided in any suitable material. The tank preferably comprises materials that reflect light within the tank, i.e. light within the tank that hits the internal side walls, bottom or light panels within the is preferably at least partially reflected back into the tank. This way, loss of light due to absorption is kept to a minimum. Accordingly, the tank preferably comprises a reflecting material on its inner surface, i.e. towards the interior of the tank. This can be achieved by using a material such as stainless steel that is naturally reflective. Alternatively, or additionally, the tank can on its inner surface comprise a reflective coating to reflect light within the tank and thereby minimize energy consumption of the photobioreactor. Such coating can additionally also serve to minimize corrosion or other chemical degradation in the tank material.

The light panels are preferably also made from material that is reflective, so as to maximize the light efficiency of the tank. Commonly used LED panels are usually made from material that reflects light. Such material can preferably be reflective in nature, contain a reflective coating and/or from a highly reflective colour, i.e. white. As a consequence, all surfaces within the photobioreactor tank can be either reflective in nature or in a colour that reflects light (i.e., white).

Thus, in an embodiment, the internal surfaces of the photobioreactor are reflective, in that the surfaces comprise reflective material. These surfaces can include the internal surface of the photobioreactor tank (e.g., walls and/or bottom) as well as internal and external surfaces of the light panels (e.g. LED panels).

As will be discussed more in the following, one advantage of the photobioreactor according to the invention is that floor space requirements for the reactor are minimized, which is a significant advantage over conventional systems currently in used, e.g., glass tubes or plastic bags. Gas inflow holes or nozzles can be located in the floor panel in the body, preferably at least along the lowermost point or segment of the body (i.e., along the trough in the floor panel in the body). In some embodiments, there can be one nozzle or hole for each chamber that is defined by illumination panels or by illumination panels and tank walls. Further gas inflow nozzles can be located for each chamber in order to achieve optimum fluid dynamics. The gas inflow nozzles may be single or multiple orifice nozzles with a single orifice direction or a multitude of orifice directions in order to influence and manage the fluid dynamics within the chambers as well as in a lateral direction between chambers. Variable gas inflow into separate chambers (i.e. between illumination panels) may also be used to create and regulate flow between chambers. Furthermore, flow management can be used to avoid biofouling in the culturing system and reduce hydrodynamic stress exerted on the algae being cultured. The gas flow into the tank can be provided by a blower (e.g., vacuum blower). The blower can be attached to, or be an integral part of, the photobioreactor or the blower can be a separate unit that can serve a single photobioreactor or alternatively multiple photobioreactors.

There can also be provided one or more inlets or nozzles for delivering carbon dioxide (CO₂) gas into the reactor tank. Such inlets can be provided separate from inlets for delivering aeration in the tank. The CO₂, which preferably should be readily dissolved in the culture so that it may be utilized, serves a carbon source in the cultivation process. Thus, it is important to deliver dissolved CO₂ into the culture for optimal growth conditions in the tank.

Inlets for CO₂ delivery can be provided in the floor panel of the body, or the inlets can be suspended into culture in the reactor.

The number and location of such inlets can be varied so as to provide the appropriate amount of desireable CO₂ flow into the reactor. For example, there can be in the range of about 2-40 CO₂ inlets in the reactor tank, such as bout 4-30 inlets or about 4-25 inlets. In the case where there is low need for CO₂ into the tank, there can be in the range of about 2-10, about 3-9 or about 4-8 inlets in the tank. For medium need for CO₂ in the tank, there can be in the range of about 8-18, 9-16 or about 10-15 inlets into the tank. For high need for CO₂ in the tank, there can be in the range of 15-40 inlets in the tank, such as about 20-30 or about 20-25 inlets.

The inlets for CO₂ delivery can conveniently be provided with diffusers that provide a stream of very small bubbles of CO₂ so as to maximize the dissolution of CO₂ in the tank. Diffusers for CO₂ delivery are known in the art, including diffusers that can be arranged in the floor and/or side walls of the body (extending through the floor and/or sides of the body) and diffusers that are designed to be suspended into the body. Any such suitable diffuser can be provided for this purpose and is contemplated to be used with the photobioreactors.

When CO₂ diffusers are provided to be suspended into the tank, the diffusers can be disposed so as to be suspended from a gas line or manifold that extends across the tank.

A CO₂ diffuser can have dimensions in the range of 20-30 mm (height/length) and 10-15 mm (width/diameter), with pores that are about 20 um.

In an embodiment, there is a CO₂ recirculation loop, into which culture from the photobioreactor tank is delivered and circulated back into the tank via one or more CO₂ recirculation loop tank outlet (PBR outlet). Since concentration of dissolved inorganic carbon (DIC) needed for optimized algal growth can be higher than achieved by injecting CO2 through an air-sparge and/or controlled by means of a pH-stat (i.e., pH control), higher DIC in the culturing liquid can be obtained via an external loop. The purpose of the loop is thus to direct culture from the tank into a closed loop, within which the culture is enriched in CO_{2,} and subsequently redirect the CO₂-enriched culture back into the tank. The loop reconnects to the tank via one or more CO₂ recirculation loop tank inlet (PBR inlet). On the CO₂ recirculation loop there is preferably at least one pump provided, for circulating culture from the tank through the loop and back into the tank. Downstream (with respect to the flow of culture) of the pump, there can be one or more CO₂ inlets in the loop, for delivering CO₂ into the loop. Downstream of the pump, there is elevated pressure due to the pumping of culture against the volume of culture in the tank. Thus, CO₂ is delivered into the culture at an elevated pressure, for maximizing the dissolution of CO₂ in the culture and thereby the conversion of CO₂ to biomass in the photobioreactor. The PBR outlet and PBR inlet that connect to the CO₂ recirculation loop can preferably be connected to the floor panel of the photobioreactor tank. It can be preferable to arrange the at least one recirculation loop tank outlet and the at least one recirculation loop tank inlet in the lower half of the body, with respect to the volume of the body (i.e. below the level of culture in the tank that would correspond to the tank be filled to half of its capacity). It can be preferable that the recirculation loop be connected to the trough in the floor panel via the PBR outlet and PBR inlet. Thus, the at least one recirculation loop tank outlet and the at least one recirculation loop tank inlet can be provided in the floor panel of the body, preferably in or near the trough.

The PBR outlet and PBR inlet can be positioned at either end of the photobioreactor tank. Thus, for example when the tank has an elongated (such as rectangular) shape, the PBR outlet can be placed near one end of the tank, preferably near or within the trough in the floor panel, and the PBR inlet can be placed near the opposite end of the tank, preferably near or within the trough in the floor panel. This way, the CO₂ recirculation loop will provide additional circulation within the tank.

In some embodiments, the PBR outlet and PBR inlet are located at or near the lowermost part of the floor panel. The PBR outlet and PBR inlet can thus be placed within a vertical distance of about 30 cm, about 20 cm, or within about 10 cm from the lowermost part of the floor panel, i.e. the lowermost point in the trough in the floor panel.

The CO₂ recirculation can furthermore contain a valve for draining fluid from the loop, and thereby from the tank. Such a valve, which can preferably be a three-way valve (i.e., a T-valve), can preferably be provided downstream of the pump on the recirculation loop. This way, when so desired, the tank can be emptied quickly using the pump to pump liquid (culture) from the tank.

In certain embodiments, the CO₂ to Biomass Conversion Factor is at least 0.2 (i.e. at least a 20% conversion of the mass of CO₂ delivered into the tank to biomass), such as at least 0.22, at least 0.24, at least 0.25, at least 0.26 or at least 0.27 external.

The biomass production per groundprint of the photobioreactor (i.e. the ground are the reactor occupies) can be expressed as the amount of protein (from biomass) produced per square meter per year. Thus the protein production of the photobioreactor as described herein can be in the range of 80 to 150 kg/m²/yr, in the range of 90 to 140 kg/m²/yr, in the range of 100 to 130 kg/m²/yr, in the range of 110 to 125 kg/m²/yr, or about 120 kg/m²/yr.

Diffusers can thus be provided in the floor panel of the body, for example at or near the bottom of the floor (e.g., in the trough in the floor panel). The diffusers can alternatively, or additionally, be provided as suspended diffusers that are suspended from above and into the culture in the tank. Illumination panels are suitably arranged within the photobioreactor tank so that culture within the tank (i.e., aqueous solutions or aqueous suspension) can flow along the sides and bottom of the tank with minimal obstruction. Preferably, there is little or no obstruction to flow of water along the side walls and floor panel of the tank, to facilitate flow along the side and bottom panel of the tank.

The illumination panels can preferably be provided as flat panels, i.e. panels that are generally flat and relatively thin compared with their length and/or width.

Panels within the tank can thus be disposed so that the panels do not meet the sides and/or bottom of the tank. This way, the panels will not impede culture (liquid) flow along the sides and/or bottom of the tank. Preferably the panels are disposed so that they do not meet either sides or bottom panel of the tank.

Alternatively, or complimentary, the illumination panels can be so provided that the panels allow free flow along the sides and bottom panel of the tank. This can be achieved by for example providing apertures in the panel structure along the side walls and/or floor panel of the tank, to allow flow of culture along the sides and/or bottom of the tank, even when the main panel structure reaches the sides and/or bottom of the tank.

The light panels can be arranged in a vertical manner within the photobioreactor tank, covering the major part of its cross-section area. The distance between light panels can be modified in order to define volumes where, in combination with gas inflow, fluid dynamics can be regulated and/or managed in a way that allows all subvolumes of the liquid in the tank to come sufficiently close to the light sources for a sufficient fraction of time to allow optimum photosynthetic activity and ripening of culture in the tank. In this way, appropriate light exposure of microbes in the tank is ensured and maximum photosynthetic efficiency of the microalgae concerned is achieved. The distance between light panels may thus be variable in order to achieve optimum fluid dynamics. The shape of light panels may also, or alternatively, be variable in order to achieve optimum fluid dynamics and/or to achieve optimal surface area or light conditions in the tank.

Thus, illumination panels can extend towards the sides and/or bottom of the tank, with slits or gaps present between the panels and the walls and floor of the body to ensure flow along sides and bottom, for efficient circulation within the tank.

The illumination panels can be disposed so that there is a gap between the panels and the side walls and/or floor panel of the body. The gap can between the illumination panels and the side walls and/or floor panel of the body can be in the range of about 2-20 cm, in the range of about 3-15 cm, in the range of about 4-15 cm, in the range of about 5-15 cm, or in the range of about 5-10 cm.

The gap may be uniform, in that the illumination panels have a shape that is parallel to the contour of side walls and floor panel in the reactor tank, and the gap as a consequence constant. Alternatively, the illumination panels may have a shape that does not follow the contour of the side walls and the floor panels precisely, and as a consequence that gap between the illumination panels and the side walls/floor panel may not be uniform.

The illumination panels can be disposed at an angle to the side walls of the body so that the angle between the illumination panel and side wall is in the range of 45° to 90°, preferably preferably 60° to 90°, more preferably 70° to 90°, more preferably 80° to 90°, more preferably 85° to 90°. Even more preferably, the angle can be about 90°, i.e. the illumination panel can be perpendicular or approximately perpendicular to the side walls. As will be apparent, by disposing the illumination panels in this fashion, the circulation of culture within the tank is relatively unrestricted and driven by air-lift in the tank.

The ratio surface of illumination panels to the volume of the tank can be adjusted, by changing the number and/or shape of illumination panels used. The higher the density of panels, the higher the ratio of surface area of the panels to the volume of the tank, and the resulting culture surface (the combined surface area of the illumination panels) per ground footprint is high.

The surface to volume ratio of illumination panels to volume of tank can be at least 5 m²/m³, preferably at least 7 m²/m³, more preferably at least 8 m²/m³, even more preferably at least 9 m²/m³, and even more preferably at least 10 m²/m³.

An advantage of placing the illumination panels within the tank is that light efficiency is maximized—the light is delivered directly into the circulating culture within the tank, and is therefore not lost through the walls (which preferably do not transmit light and preferably also reflect light on their internal surface) or the top of the photobioreactor.

The energy consumption in the tank can be in the range of 500-2000 kWhr/kg biomass, preferably in the range of 700-1800 kWhr/kg, more preferably in the range of 900-600 kWhr/kg, more preferably in the range of 1100-1400 kWhr/kg, more preferably in the range of 1200-1300 kWhr/kr.

The body or tank of the photobioreactor can have a generally elongate shape, i.e. its width can be smaller than its length. The body can be generally rectangular when viewed from above. Alternatively, the body can have an oblong, ellipticial or circular shape when viewed from above.

It can be preferable that the trough in the floor panel of the body be such that the trough extends longitudinally along the floor panel. The trough can for example extend longitudinally along the middle of the floor panel.

The floor panel can preferably contain at least one convex segment to define a trough (the lowermost part of the floor panel), towards which debris in the tank will flow. The floor panel can thus have a generally convex shape. In an embodiment, the floor panel comprises a continuously downwardly convex structure, so that debris can flow towards the lowermost part of the floor panel. The shape of the panel can be symmetrical, so that when viewed end-on, the panel comprises a symmetrical convex structure.

For example, the floor panel can have a generally half-cylindrical shape, so that the trough in the floor panel is defined by the bottom of the half-cylindrical structure. Such half-cylindrical shaped floor panel can, when viewed end on half a semicircular cross-section, i.e. a cross section that forms half a circle.

The photobioreactor-volume pr. square meter footprint can be in the range of 0.6-1.4 m³/m², preferably in the range of 0.7-1.3 m³/m², more preferably in the range of 0.8-1.4 m³/m². Including workspace around each reactor, i.e. floor space needed to access each photobioreactor, the ground footprint can be in the range of 0.36-0.84 m³/m², preferably in the range of 0.42-0.78 m³/m², more preferably in the range of 0.48-0.84 m³/m².

The photobioreactor can be provided in a variety of sizes and shapes. It can be preferable to provide the photobioreactor with relative dimensions of its body so that its width, at its widest point, to its height, is in the range of about 0.5:1 to about 2:1, preferably about 0.8:1 to about 1.5:1, more preferably about 0.9:1 to about 1.2:1, even more preferably about 1:1.

Further, the side walls of the body can have a length in the range of about 0.5-15 m, in the range of about 1-10 m, in the range of about 2-8 m, in the range of about 3-7 m, or in the range of about 4-6 m. The end walls of the reactor tank can in general be in the range of about 0.3-4 m, in the range of about 0.5-3 m, in the range of about 1-3 m or in the range of about 2-3 m.

It will be appreciated that the reactor can be adapted to have dimensions that consist of any combination of the above recited lengths and width of the walls of the body.

The photobioreactor can be provided with one or more water inlet and one or more water outlet, for delivering water/culture/nutrients into, and draining water from, the body of the reactor. There can be one or more filter or filter systems arranged on, or in fluid communication with, the water inlet or alternatively on pipes that lead toward the one or more water inlet. Such filters serve the role of minimizing risk of contamination from the water source used to feed water into the tank. There can be separate inlets for nutrients and cell culture and/or water into the tank. When so arranged, there can be separate filters on nutrient inlets and cell culture/water inlets. One advantage of the photobioreactors according to the invention is high productivity. The photobioreactors can, during culture growth, thus result in volumetric productivity that is, in terms of culture dry mass produced per volume of culture per time unit, at least 0.2 g/L/day, preferably at least 0.5 g/L/day, even more preferably at least 0.4 g/L/day.

As a result of the high productivity and high surface to volume ration, the photobioreactors result in high productivity per unit ground area. Thus, the photobioreactors can result in areal productivity, measured in terms of mass culture per unit ground area per unit time, that is at least 300 tons/hectar/year, preferably at least 550 tons/hectar/year, more preferably at least 300 tons/hectar/year, even more preferably at least 900 tons/hectar/year.

Illumination panels may be flat and rectangular or have other shape to fit the tanks. For example, if the tank has a semiannular floor or bottom, the illumination panels may suitably have outer edges that are at least partially semiannular. Light sources may disposed on one side or both sides of boards or panels that are protected by water-proof, optically transparent material. The illumination panels can be easily removed from the tank for cleaning purposes, if preferred. The water-proof, optically transparent materials is preferably of polymeric nature. Alternatively, the material can be a suitable type of glass. An important property of the water-proof material is, that it can be easily cleaned and disinfected using routine methods.

Illumination provided by the light panels can adapted in various ways. Thus, light intensity and characteristics can be modified through one or more of (i) density of lights (bulbs/chips) in the light panel; (ii) density of light panels in the tank; (iii) intensity of light in the panels, (iv) frequency of light emittance (v) wavelength of the emitted light. Thus, for example, it can be advantageous to intermittently flash light so that there are brief intervals of light emission with dark periods spanning those intervals. Such light intervals can for example be in the range of about 1 ms to about 1 s.

The illumination panels can be controlled individually, i.e. the light of each of the panels can be controlled independently of other panels. Alternatively, the illumination panels are controlled uniformly, i.e. by setting the light emitted by each panel to be equal to that of all other panels.

The light can also be adapted based on the microbial culture in the tank. Light requirements by microbes varies considerably—to meet these requirements, the various parameters (density of light, intensity, wavelength) can be adapted as needed.

The light intensity can also be adapted to the density of the culture in the tank. Thus, as the culture density increases, light intensity in the tank can be increased to meet the decreased transmission of light in the aqueous culture.

Furthermore, light intensity can be adapted depending on the product of interest. For example, optimal astaxhanthin production by H. pluvialis requires high-intensity light. Therefore, it may be advantageous to use a relatively high density of light panels in the tank and/or operate the panels so as to emit high intensity light when growing H. pluvialis for astaxhanthin production. During growth of other microbes, light parameters and density of light panels can be varied as needed.

The illumination panels can comprise light sources that are adapted to provide illumination of any suitable intensity and wavelength. The light sources can suitably be a light source that emits visible light. The light sources can also, or alternatively, be adapted to deliver light of particular wavelengths. For example, the light sources can be adapted to deliver blue light at a wavelength of about 450 nm and/or red light with wavelength at about 640 nm and/or light in the UV and near-infrared range.

An illumination panel can comprise a plurality of light sources that can be adapted to deliver light of different spectral qualities. Thus, by using one set of light sources, light emitting particular wavelength range or of a particular wavelength can be used during vegetative growth, while using a second set of light sources within a panel in order to manipulate the cultivated organism for production of secondary metabolites of economic interest. Different panels within a tank can also suitable contain an identical or different arrangement and selection of light sources, as deemed appropriate for the particular use.

The light intensity delivered by the light sources can suitably be characterized by a photon flux that is in the range of 20-1200 μmol/m²/s, such as about 50-800 μmol/m²/s, about 80-600 μmol/m²/s or about 120-500 μmol/m²/s.

In some embodiments, the light intensity delivered by the light sources can be set within a first range during a first growth period, followed by a setting to a setting within a second range during a second growth period. For example, during growth of light-sensitive algae such as Haematococcus pluvialis, it can be useful to grow the algae in a first growth period (a “green period”) at a first setting of light intensity that is within a first range, and during a second growth period (a “red period”), the algae can be grown at a second setting of light intensity, that is within a second range that is different from the first range. For example, the first range of light intensity can be in the range of 20-400 μmol/m²/s, such as a range of 50-400 μmol/m²/s, such as a range of 100-300 μmol/m²/s. The second range of light intensity can be a range of 500-1200 μmol/m²/s, such as a range of 600-1000 μmol/m²/s, or a range of 600-900 μmol/m²/s.

Changes in light intensity can be achieved by adjusting the light intensity emitted by individual light sources or by altering the number of light sources that emit light, or both. For example, when using LED panels, a first setting of light intensity can be achieved by turning on a first subset of the LED lights within a LED panel, and a second setting of light intensity can be achieved by turning on a second subset of LED lights within the LED panel. To reduce the space needed for illumination panels as well as to reduce potential heat generation, illumination panels can comprise LED or OLED lights that are known in the art. For example, the panels can each comprise one or more LED or OLED light boards or light engines, wherein each such light board comprises a plurality of lights that are embedded on a circuit board that has electrical and mechanical fixings. Exemplary LED light boards (light engines) are manufactured by Light Engines Europe (http://www.leeltd.uk.com).

Individual light boards can be linear (comprising a single row of LED lights), circular or rectangular. Within each illumination panel there can a plurality of such light boards, for example a plurality of rectangular light boards, a plurality of a combination of rectangular light boards and linear (or strip) light boards, a plurality of a combination of rectangular, linear and/or circular light boards, etc., as appropriate for each particular illumination panel, so that internal light boards in the panel cover a substantial proportion of its planar area.

Illumination panels can comprise a housing, within which one or more light board is arranged. The housing is preferably water-proof and made from light-transparent material, such as poly(methyl methacrylate) (PMMA), also known as acrylic glass, examples of which include Plexiglas, Acrylite, Lucite, Crylux and Perspex, so that light that is emitted by the light boards can penetrate the housing and illuminate culture in the tank. The housing can for example comprise, or consist of, a thin largely planar (flat) structure of sufficient thickness to be able to accommodate light panels. It can be convenient to arrange a plurality of spacers between light boards within the panel, to prevent swaying or deflection of the panel due to water pressure within the tank. There can also, or alternatively, be one or more spacer arranged between or around light boards in the illumination panel, to preserve its structural integrity during use.

Illumination panels can also comprise, or consist of water-proof light boards, i.e. light boards that can operate functionally when immersed in water. The illumination panel can thus comprise a single such water-proof light board. The illumination panel can alternatively comprise one or more such water-proof light boards that are secured to a mounting frame that is inserted into the body. The mounting frame can comprise any suitable structure that can securely hold light boards in place. For example, the mounting frame can consist of, or comprise, one or more mounting panel, which can preferably be made from light-transparent material, and on which one or more light board is mounted.

Water-proof light boards can also comprise light boards that have been embedded in a water-proof, light-transparent material, such as suitable polymeric plastic material. Such embodiments can comprise one light board embedded in a light-transparent material. Alternatively, a plurality of light panels can be embedded together, so as to form a single illumination panel that consists of a plurality of embedded light boards.

Gas inlet into the tank within the trough can be provided by gas inlets that extend through the floor of the tank. The gas inlets can also, or alternatively, be provided by gas dispersion means that is provided internally in the body, within the trough in the floor panel. Such gas dispersion means can for example consist of a plurality of gas inlets provided on a gas line that extends along the trough in the floor panel.

The gas inlets provide a stream of gas (e.g., air) that provides aeration and air-lift within the tank. The gas flow can be adjusted so as to generate the desirable degree of aeration/air-lift in the tank, depending on e.g. (i) the dimensions of the tank, (ii) the number of gas inlets in the tank, and (iii) the need of the particular culture in the tank.

The gas inlets can comprise spouts for gas delivery. Preferably, the spouts extend no more than 10 mm into the body of the photobioreactor, preferably no more than 8 mm, no more than 5 mm, and most preferably no more than 3 mm into the body.

The gas inlets can have an internal diameter that is in the range of about 1-10 mm, preferably in the range of about 2-8 mm, more preferably in the range of about 2-6 mm, more preferably in the range of about 3-5 mm.

The gas inlets can be arranged uniformly within the floor panel of the body. The gas inlets can be provided on or along the lowermost section of the floor, so as to facilitate recirculation of culture within the tank from the lowermost parts of the tank floor. It can thus be preferable to arrange gas inlets along the trough in the floor panel. When so provided, the gas inlets can be provided at regular intervals. It can be preferable to arrange the gas inlets so that they are located in between illumination panels in the photobioreactor (when viewed from above). In an embodiment, gas inlets are provided so that there is at least one gas inlet located in the floor panel, in between each illumination panel in the photobioreactor when viewed from above.

There can be a plurality of generally parallel, linear arrangements of gas inlets in the floor panel of the body. Preferably, one of such linear arrangements extends along the bottom of the trough in the floor panel.

For example, the gas flow can be in the range of about 50 L/m³/min to about 500 L/m³/min, such as in the range of about 100 L/m³/min to about 400 L/m³/min, in the range of about 200 L/m³/min to about 300 L/m³/min. In some embodiments, the gas flow is about 200 L/m³/min, about 250 L/m³/min, or about 300 L/m³/min.

The photobioreactor in accordance with the invention can have an open top, or the top can be closed. It may for example to be desirable to close the photobioreactor tank during growth to minimize risk of contamination into the tank and/or to maintain a sterile environment (i.e. avoid contamination by undesirable microorganisms in the tank). Another advantage of closing the tank is that loss of water due to evaporation is kept to a minimum. Thus, it is contemplated that the photobioreactor in accordance with the invention be provided by means for closing the photobioreactor when so desired.

For example, means for closing the photobioreactor can be provided by a single continuous lid structure that covers essentially all of the tank, resulting in a tank that is essentially a closed system, i.e. a system that is not open to the outside except through controlled valves and other inlets or outlets that are arranged in the photobioreactor and its tank. There can also be two or more lid panels provided, the panels being arranged on the photobioreactor top so as to cover the top. The panels can preferably comprise a rectangular structure. The individual panels can have an essentially flat upper surface. Alternatively the panels can be slanted along at least a portion of their surface. The panels can be slanted from one edge to the next. The panels can alternatively be slanted from a middle portion thereof, towards two or more edges of the panels.

An advantage of having a lid on the tank is that due to the tank being closed, evaporation from the tank is minimized; evaporated water condenses on the inner surface of the lid, and does not escape from the tank.

There can be a plurality of nozzles or inlets provided on the panels. The nozzles or inlets serve the role of allowing for delivery of e.g. a rinsing or washing solution through the lid and into the tank, without having to remove the lid, or one or more lid components. The nozzles or inlets can therefore be connectable to tubes, pipes or manifolds for delivering rinsing or washing solutions through the lid and into the tank. Preferably, the nozzlets or inlets extend through the lid, so that rinsing/washing solutions can be delivered through each nozzle or inlet and into the tank.

### BRIEF DESCRIPTION OF THE FIGURES

The skilled person will understand that the figures, described below, are for illustration purposes only. The figures are not intended to limit the scope of the present teachings in any way.

FIG. 1 depicts a photobioreactor in accordance with the invention that is enclosed by an outer frame.

FIG. 2 shows a cross-sectional view of the photobioreactor shown in FIG. 1, showing the internal body and panels within the body.

FIG. 3 shows a longitudinal cross-sectional view of the photobioreactor shown in FIG. 1, showing internal panels along the body of the container, as well as air inlets in the floor panel of the body.

FIG. 4 shows a perspective view of a modified photobioreactor according to the invention.

FIG. 5 shows a longitudinal cross-sectional view of the photobioreactor depicted in FIG. 4

FIG. 6 shows a cross section of a photobioreactor as illustrated in FIG. 4, showing internal panels and a cross-section view of drivers disposed on the outer side walls of the reactor.

FIG. 7 show in (A) and (B) show respectively two alternate photobioreactors in accordance with the present invention.

FIG. 8 shows another photobioreactor in accordance with the invention having a tubular shape.

FIG. 9 shows how CO₂ diffusers can be arranged in the floor panel of the photobioreactor tank.

FIG. 10 shows a photobioreactor comprising a manifold assembly for disposing suspended CO₂ diffusers into the photobioreactor tank.

FIG. 11 shows how a schematic illustration of the circulation within the photobioreactor tank that is generated by gas delivery into the bottom of the tank floor.

FIG. 12 shows an illumination panel in accordance with the invention.

FIG. 13 shows an alternative illumination panel in accordance with the invention.

FIG. 14 shows an exemplary thermal jacket that can be provided on the outer surface of the photobioreactor tank.

FIG. 15 shows a CO₂ recirculation loop that can be connected to the photobioreactor tank.

FIG. 16 shows a photobioreactor with a two-part lid arranged thereon, to close the photobioreactor tank.

FIG. 17 shows an alternative lid arrangements, where six lid units, each having a slanted upper surface, can be used to close the photobioreactor tank.

### DESCRIPTION

In the following, exemplary embodiments of the invention will be described, referring to the figures. These examples are provided to provide further understanding of the invention, without limiting its scope.

In the following description, a series of steps are described. The skilled person will appreciate that unless required by the context, the order of steps is not critical for the resulting configuration and its effect. Further, it will be apparent to the skilled person that irrespective of the order of steps, the presence or absence of time delay between steps, can be present between some or all of the described steps.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise. The present invention provides a productive, sustainable and resource-efficient (energy, space and water) production system, allowing for mass culture and exploitation of valuable micro-algae derived bioactive compounds. The system provides for not only a low, but a negative carbon footprint production chain, as the algae culture consumes CO₂ and delivers back oxygen. The photobioreactor is furthermore designed for integrated pest management to considerably reduce product losses and waste on algae farms. Present culturing techniques are expensive, and producers indicate to lose ⅓ of their harvest because of pests, usually unwanted microalgae species that take over the production systems.

Thus, the system and method is expected to play a crucial role in unlocking the cost-efficiency of microalgae cultivation systems so that microalgae cultivation can eventually be efficiently used for production of food and bio-based products, even in unfavourable climates.

There is a general unmet demand on the market for industrial sized tanks for algae culturing. Current solutions utilize tubular glass reactors, including those offered by Varicon Aqua Solutions. Small non-industrial sized tanks are offered by two companies, Algae Food & Fuel (1000 L tank-based PhotoBioReactor for laboratory use) and Industrial plankton in Canada (1250 L tank-based PhotoBioReactor). Both these systems are small and unpractical for farmers; furthermore, Industrial Plankton's solution is very expensive.

The present invention has been made with an aim of solving problems of limited and uneven access of light-sensitive microbes, such as certain algae, to light in photobioreactors and of problems with cleaning and disinfection of photobioreactors to avoid contamination, both problems having a negative influence on yield. The invention provides tanks having particular structure comprising immersed illumination elements or panels that are mounted in the tank at certain intervals. One advantage of the invention is that the lighting elements can easily be removed for cleaning, if needed. The photobioreactor can be scaled up by serial connection of tanks in a modular way. Furthermore, tank sizes can be varied as needed, increasing the flexibility of the solution.

Nevertheless, the skilled person will appreciate that an important practical advantage is that the photobioreactor described herein is a stand-alone system, in that it does not require additional functionalities, supply tanks, harvesting tanks or external components for its use. Moreover, the photobioreactor can be operated in a continuous fashion without having to remove any components during or in between culturing cycles. If so desired, the photobioreactor can in fact be kept closed at all times, with no requirement for opening the tank in between cycles, for draining, cleaning or other reasons.

The photobioreactor according to the invention is based on an inverted panel configuration, where the light panels are enclosed and disposed within a tank for culturing e.g. light sensitive microbes, delivering light into the tank at regular intervals within the tank. This is an inverse configuration compared with prior art solutions, where the panel encloses the algal culture and the light source is positioned outside the panel. The unique configuration of the invention significantly enhances the operational efficiency of the culturing system by allowing operation and service of large culture volumes in a single unit while still receiving light from numerous light panels which in turn simplifies and speeds up the culturing process. This is made possible by using flat panels that are arranged at an angle to the side walls of the tank. The flat panels can in particular be disposed within a tank having a floor that is sloping, or has at least one sloping section, so that culture within the tank flows toward the bottom of the floor, which will be in the shape of a trough in the floor (or floor panel). By providing airlift from the trough in the floor panel, efficient aeration and circulation of microbes in the tank is provided which, when combined with the high density lighting capabilities, provides a photobioreactor that has significant advantages over prior art solutions.

An inverted layout of the flat-panel principle is thus a new technical approach where the light source is positioned in thin panels submerged into and surrounded by a culturing liquid receiving photosynthetic active irradiation from the source for driving growth. The culture is enclosed by a tank with a sloping floor (preferably U-shaped, or having a U-shaped lower portion) and the culture is mixed and circulated in the tank by air injected through sparges located in in the bottom of the tank.

The present invention solves i.a. the problem of uneven exposure of light-sensitive culture to light by applying a combination of tank shape, nozzle arrangement and/or orifice direction and defined illumination panel structure and illumination panel intervals to manage the fluid dynamics in the tank and create flow characteristics that ensure even light exposure to all subvolumes of the liquid culture. The present invention at the same time solves the problem of cleaning and disinfection by creating easy access to all exposed surfaces, tank surfaces as well as light panel surfaces. The interior of the tank, including all internal surfaces can thus be easily cleaned, without removing any components of the tank for cleaning. The internal surfaces of the tank are thus smooth and easily accessible for cleaning.

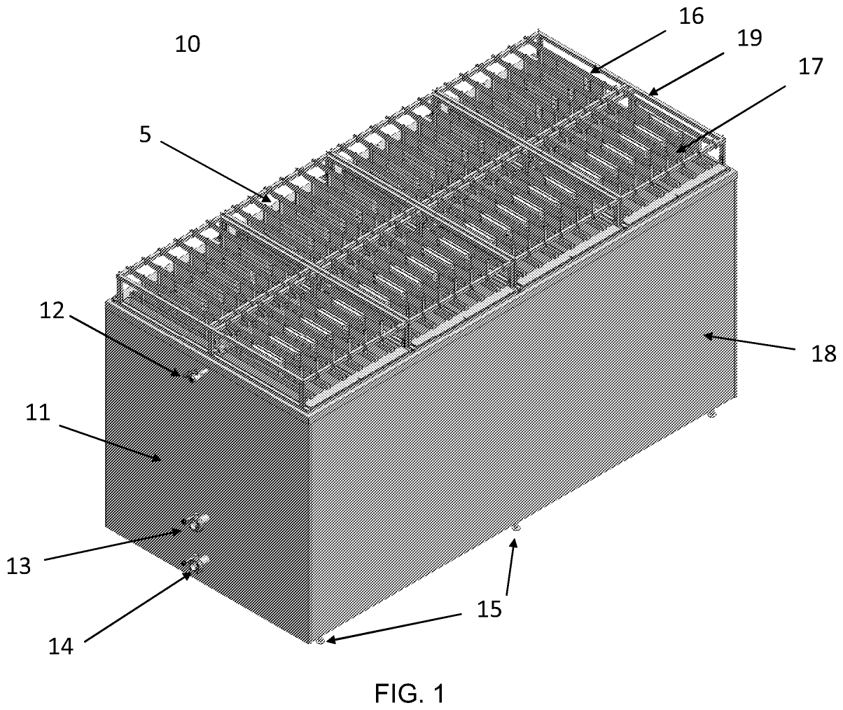

Turning to FIG. 1, there is depicted a photobioreactor 10, comprising an inner body 5 (not seen except from above in this view) enclosed by a rectangular outer frame structure consisting of side walls 18 and end walls 11. The end walls can be separate from, and disposed outwardly from, the end walls of the body 5 of the photobioreactor, such that the entire body is enclosed within the outer frame structure. Alternatively, the end walls 11 may also serve as end walls of the internal body 5, such that the end walls also represent a portion of the outer frame structure. The frame structure is in such embodiments completed by side walls 18, which may be in the form of side panels 18 that are arranged on a common frame. Legs 15 extend downwardly from the side walls 18, providing support to the outer frame structure.

A plurality of illumination panels 17 is disposed within the body 5. The illumination panels are arranged in parallel so as to be suspended via supports 16 that sit on upper rims on a frame structure 19 disposed on the upper rim of side walls of the body 5 or side walls of the photobioreactor 10. In the embodiment shown, there are eight sets of illumination panels 17 shown, each set containing six illumination panels that are arranged in tandem within a section of the frame structure 19. Four such sets of tandem arrangements are provided along each sidewall 18 of the photobioreactor, so that overall there are in total 48 illumination panels 17 in the reactor. The illumination panels 17 are removable from the frame structure 19. Thus, each panel can be removed as needed, e.g. for maintenance or cleaning purposes. Furthermore, the density of illumination panels 17 can be modified in accordance with the particular culturing needs. Thus, either fewer or more illumination panels can be arranged on the frame structure 19.

Valves 12, 13, 14 are arranged on lines extending from the body 5 and through the end wall 11. On each such line, there is a valve 12,13,14 provided, to control flow through line, to deliver fluid into the body 5, or to drain fluid from the body 5. Lowermost outlet 14 and/or outlet 13 can conveniently be used for draining the body 5, and fluid (e.g. water, nutrients and/or liquid culture) can be added through upper valve 12. However, as should be appreciated, fluid (water, nutrients, culture) can also, or alternatively, be added to the tank via the lower lines into the tank, regulated by valves 13,14.

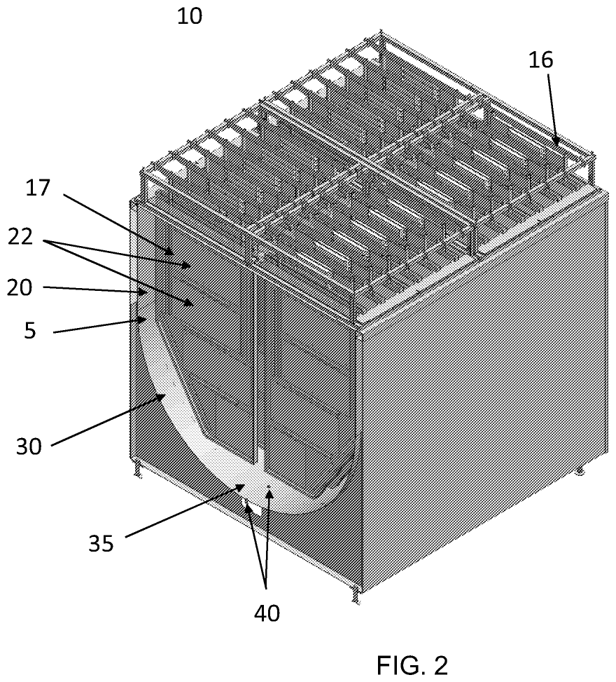

A cross-sectional view of the photobioreactor in FIG. 1 is shown in FIG. 2. The body 5 can be seen to be defined by side walls 20 and floor panel 30. The floor panel has 30 a generally half-tubular shape, i.e. the floor panel has a semi-circular cross section such that the cross section is in the shape of a half circle. Trough 35 is formed by the lowermost portion of the floor panel. Due to the shape of the floor panel, debris of solid particles within the tank will flow towards and accumulate within the trough 35.

At the bottom of the trough 35 there are gas inlets 40 arranged along the trough. The gas inlets 40 are arranged at regular intervals along the trough 35. When the tank is filled with a liquid culture, gas (e.g., air) that is pumped into the body 5 via the gas inlets 40 will generate air lift in the tank, driving microbes being grown in the tank towards the liquid surface within the body. Natural flow of such airlift is toward the sides, thus driving the culture towards the side walls 20 of the body 5. Once the microbes reach the side walls, they will gravitate downwards, in the direction of trough 35 in the bottom of the body 5. Naturally, there will also be gravitational and random (Brownian) motion of liquid in the tank such that there will be a downward flow not only along the side walls, but also throughout the body, away from the side walls and floor panel. However, the overall effect of the airlift generated through air pumped through gas inlets along the middle of the floor panel of the body and the gravitational flow along the side walls towards the trough 35 will be that of circulation and mixing of culture within the tank, as further illustrated.

Circulation within the body is aided by liquid flow along the sides and bottom panel of the tank. Thus, in the shown embodiment, illumination panels 17 can be seen to not reach the side walls 20 or floor panel 30 of the body 5. Thereby, there is unrestricted (free) flow of liquid culture along the sides and the floor panel of the body 5, aiding in the unrestricted flow and circulation within the body.

The illumination panels 17 are provided by internal light sources (LED lights), provided by four LED panels 22 arranged within each illumination panel 17. Circulating culture within the tank will therefore come into contact with light emitted by the illumination panels. The degree and timing of illumination can be adjusted by both the degree of circulation (driven by air pumped into the tank via gas inlets 40) and the strength and wavelength of light emitted by the illumination panels, as well as the density of panels (distance between panels) in the body 5. Light intensity can also, or alternatively, be adjusted during growth, as light transmittance is significantly decreased with increased culture density.

Circulating light sensitive cultures in the tank (e.g. H. pluvialis) will thus experience high intensity light (high light exposure) when in close distance to the illumination panels 17, while when further away from the light (low light exposure), the culture will not experience such conditions. In other words, there will be a constant shift in exposure from low to high light exposure. In the case of H. pluvialis, high light exposure triggers biosynthetic pathways leading to astaxanthin formation in the cells. During conditions of astaxanthin formation (“red” phase) it can therefore be advantageous to grow H. pluvialis at high density of light panels and/or high light intensity, while other microbes may require less light exposure.

If desired, the wavelength of the light can be adjusted during culturing, so as to promote selective growth during the red and/or green phase of the culturing.

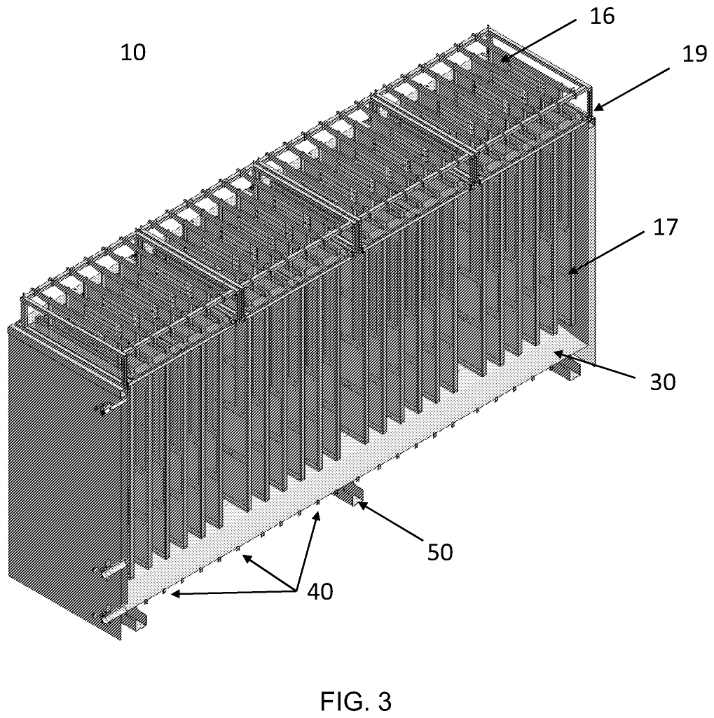

In FIG. 3, there is shown a longitudinal cross-sectional view of the tank 10. A plurality of gas inlets 40 can be seen to be disposed along and through the lowermost portion of the trough 35 in the floor panel 30. The gas inlets thus extend along the lowermost section of the tank, so as to provide an airlift from the bottom of the tank. This regular arrangement of gas inlets provides for uniform airlift along the longitudinal axis of the tank.

As an advantage of the uniform airlift in the tank is that the culture within the tank is well and uniformly mixed at all times. Following the addition of a solution (for example a starter culture) to one end of an otherwise filled tank, the solution within the tank reaches a homogeneous composition within about 15 minutes, a consequence of the rapid and thorough mixing provided by the airlift of the gas (e.g., air) delivered into the tank via the gas inlets 40 in the floor of the tank.

Three support members 50 provide support to the photobioreactor when placed on a flat surface. Additional support members can be provided as required, depending on the load on each tank, which depends on its length and height.

The photobioreactor also comprises one or more CO₂ inlets (not shown in this view), for supplying a stream of CO₂ gas into the tank, which serves to provide a source of carbon and adjust the pH in the tank. There can further be controllers arranged on the tank, to control growth parameters in the tank, including for example pH, temperature, inorganic salt levels, and illumination conditions.

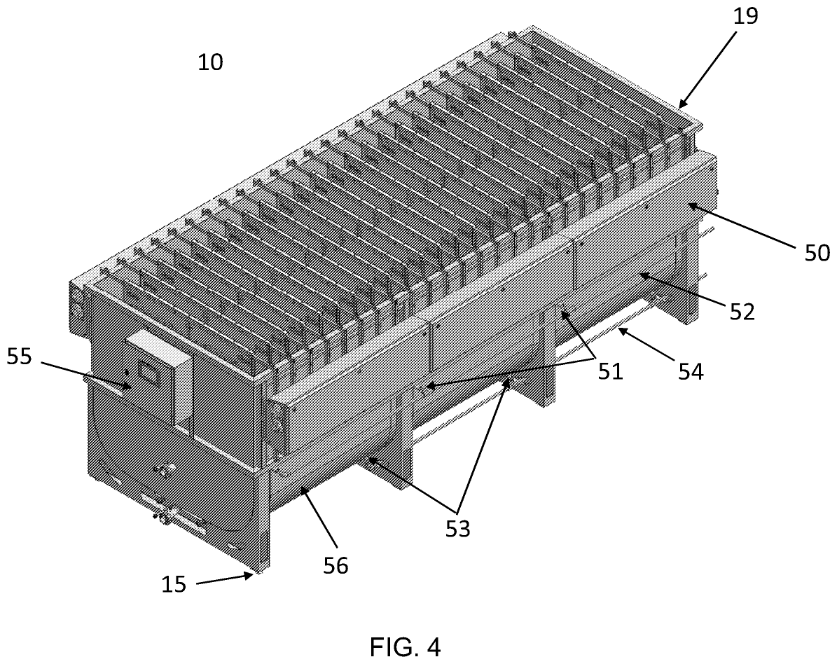

In FIG. 4, there is shown a perspective view of an alternative photobioreactor in accordance with the invention. Referring to this figure, there is a control box 50, containing electronic controllers for controlling valves, illumination panels, pH, temperature and/or other parameters in the tank. The controllers can be manual or automatic, or a mixture thereof. For example, there can be controllers that are adapted to automatically respond to certain parameters, for example measurements of temperature or pH, and adjust conditions in the tank accordingly, depending on preset parameters.

The tank in the photobioreactor can be double walled, to define an open space (thermal jacket) surrounding the tank that can be filled with a fluid such as water, for controlling temperature in the tank. The outer surface of the double wall of the tank 56 can be seen. A series of inlets 53 are arranged in the outer wall of the tank. Water line 54 leads into these openings, for delivering water into the thermal jacket (line 54 and inlet 53 are shown disconnected in this view). Water exits the thermal jacket through exit openings 51, which can be connected to exit water line 52 (the exit openings and exit water line are depicted to be disconnected in this view).

The depicted tank has three entry openings and three exit openings, for delivering water into and out of the thermal jacket, respectively. However, it will be appreciated that fewer or additional such openings can be accommodated as desirable, depending on the dimensions and configuration of each tank. The electronic controller 55 can receive information about temperature in the tank from a temperature probe that can for example be suspended into the tank. The controller can compare measurements of the actual temperature to a set temperature, and adjust the temperature and/or water flow through the thermal jacket as needed, to bring the measured temperature in the tank to that of the preset temperature. If the temperature in the tank is too low, warmer water can be circulated through the thermal jacket. Alternatively, the controller may adjust flow through the thermal jacket to a higher setting, so as to bring fluid in the tank more quickly to the desired temperature.

Boxes 50 on the side of the photobioreactor structure contain power supplies to drive illumination in the illumination panels. The boxes 50 furthermore contain electrical switches for the lights in the illumination panels.

The photobioreactor can have dimensions so that its internal volume (the volume of the body) is about 7500 L. Each illumination panel in the photobioreactor provides illumination over an area of about 2.9 m². The reactor has 25 illumination panels, for an overall area of illumination of about 73.2 m². This means that overall, the illumination are per unit volume is 0.00976 m²/L or 97.6 cm²/L.

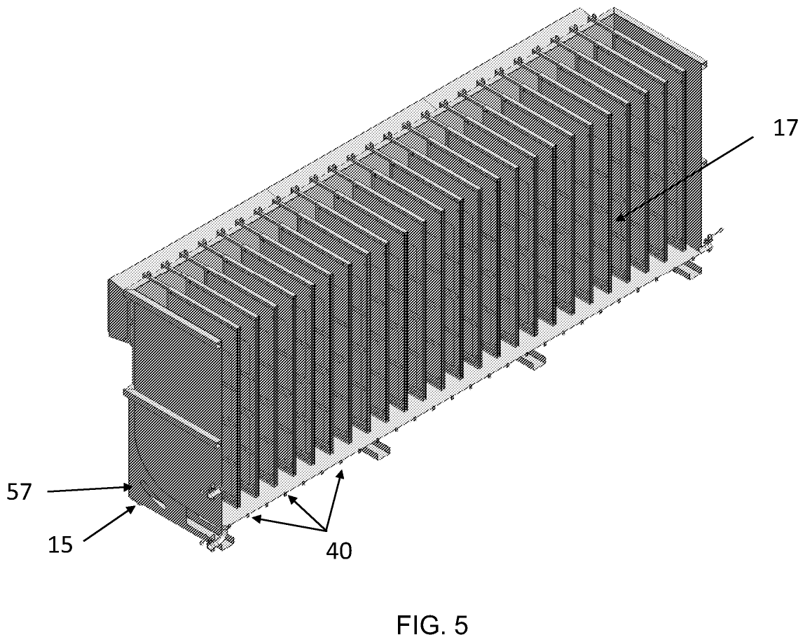

A longitudinal cross-section of the tank in FIG. 4 is shown in FIG. 5, illustrating the relative position of illumination panels 17 and gas inlets 40 along the trough in the tank floor. In this view, it can also be seen how frame member 57 provides support to the tank, and legs 15 extending downwardly from frame members 57.

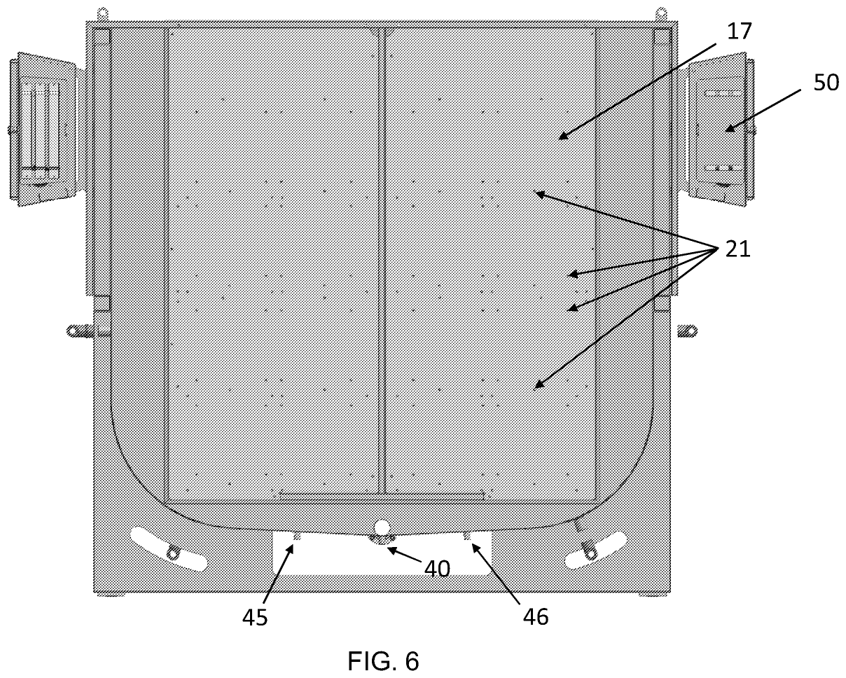

In the cross-sectional view shown in FIG. 6, there are additional gas inlets 45, 46 shown, in addition to gas inlets 40. Air flow through gas inlets 40 can be supplemented by additional air flow through gas inlets 45, 46, thereby providing additional airlift in the tank. Also shown in FIG. 6 is a side view of illumination panels 17, with a plurality of LED lights 21 being disposed within each of the illumination panels. The LED lights are powered by power supplies (drivers) within boxes 50, and controlled by controller 55.

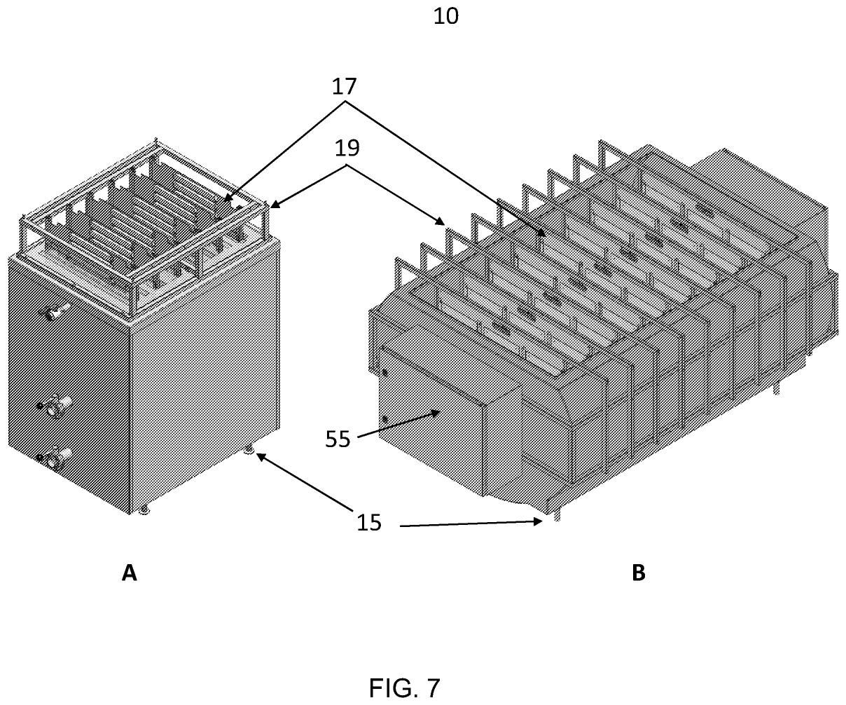

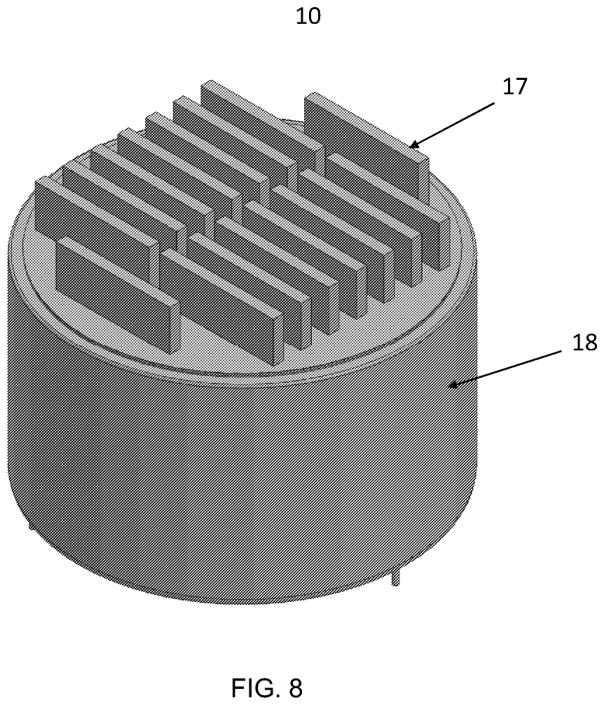

Alternative designs of photobioreactors in accordance with the invention can be seen in FIG. 7 and FIG. 8, respectively. Thus in FIG. 7 there are shown in (A) and (B) two alternative photobioreactor designs, each showing relatively small photobioreactors utilizing the same overall concept, i.e. each photobioreactor comprises a tank that contains illumination panels in an inverse configuration in that the panels are disposed within the tank. Thus, the photobioreactor shown in (A) contains illumination panels 17 suspended within a tank 10 in an analogous fashion to the reactor shown in FIG. 1. A wider configuration of a photobioreactor is shown in (B), where a series of illumination panels are controlled by means of controllers arranged within a control box 55.

Yet another design, shown in FIG. 8, shows a photobioreactor 10 comprising a round tank structure, where the outer side wall 18 surrounds the body of the photobioreactor. Illumination panels 17 can be seen to be suspended into the tank, in an analogous fashion to that shown for other embodiments in accordance with the invention.

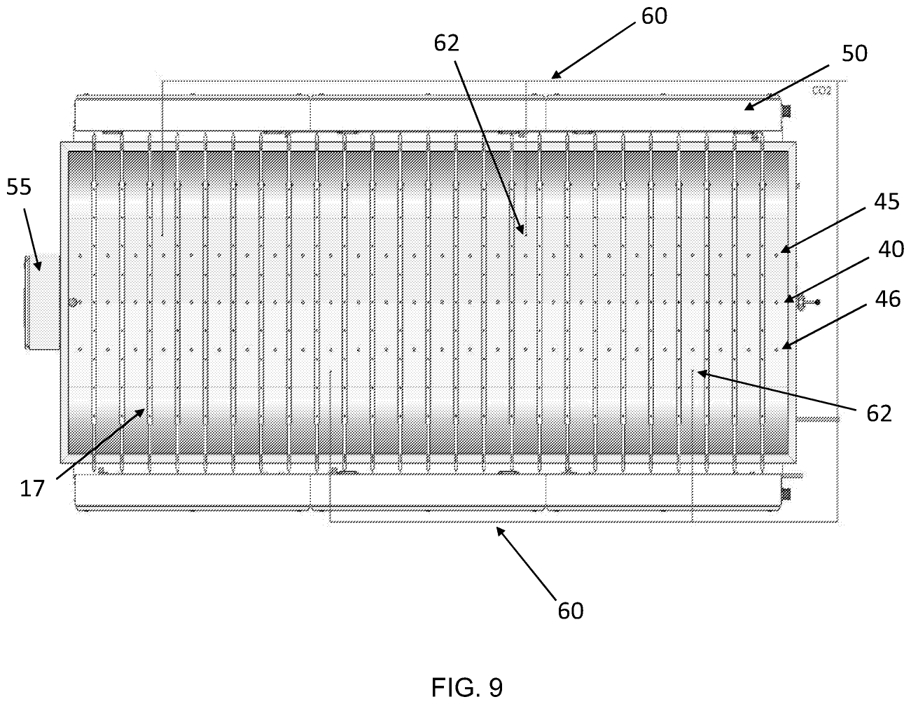

Inlets for delivering carbon dioxide (CO₂) can be provided in the photobioreactor, for controlling pH within the photobioreactor tank. In FIG. 9 it is shown how gas lines for delivery of CO₂ can be provided in the tanks. A stream of CO₂ is delivered through gas lines 60, and delivered into the photobioreactor via gas inlets 62. The gas inlets are preferably provided as a diffuser, that has very small pores (typically about 20 μM) so that CO₂ gas is dispersed by very small air bubbles into culture in the tank. This is important so that the introduced gas dissolves in the aqueous culture in the tank.

In the top view of FIG. 9 it can be seen that CO₂ inlets 62 are provided in the floor of the container, close to gas inlets 45,46 that provide airlift in the tank. The stream of CO₂ gas provides additional and supplementary agitation and airlift in the tank. A series of gas inlets 40,45,46, for providing airlift in the tank are shown to be provided in between illumination panels 17 in the tank.

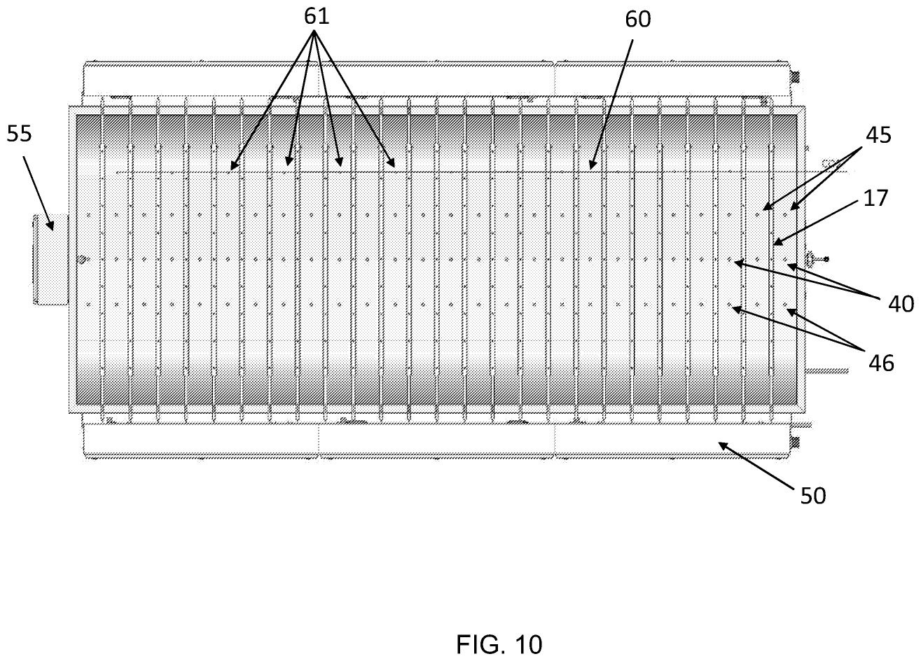

Alternatively, CO₂ can be delivered into liquid culture in the tank via diffusers that are suspended into the tank from above. An example of such a configuration is shown in FIG. 10, where a plurality of diffusers 61 are indicated to be suspended into the tank via CO₂ gas line 60 that is disposed above the body of the photobioreactor.

By providing CO₂ into the tank from above via suspended diffusers, the position (height) of the diffusers in the tank can be adjusted as needed.

Irrespective of the configuration of CO₂ delivery into the photobioreactor, the number of CO₂ diffusers can be altered and/or the stream of CO₂ into the tank adjusted so as to provide an appropriate adjustment of dissolved CO₂ (resulting in control of pH) in the tank. The stream of CO₂ can be automatically controlled by controller 55, which receives input about pH in the tank (obtained either manually or automatically), and adjusts the stream of CO₂ into the tank based on such measurements. Alternatively, there can be manual adjustment of the stream of CO₂ into the tank based on pH measurements of the culture within the tank.

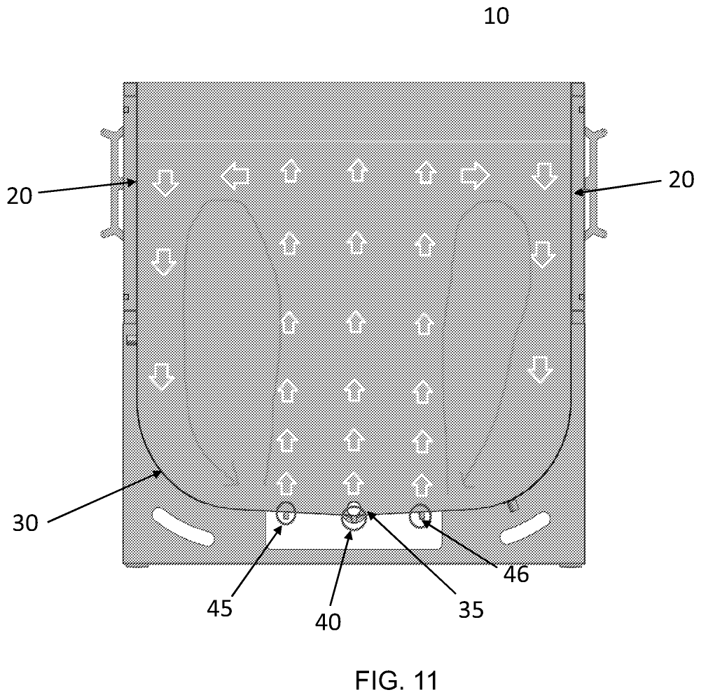

A schematic illustration of the airlift generated in the photobioreactors in accordance with the present invention is shown in FIG. 11. A series of gas inlets 40,45,46 are shown in this cross-sectional view. As illustrated in FIGS. 9 and 10, such inlets can be provided at regular intervals in the tank, for example in between each illumination panel 17 in the tank.

The stream of gas (e.g. air) into the trough 35 in the floor section of the tank through inlets 40,45,46 drives water in the tank, and thereby also culture that is suspended in the tank, towards the culture surface in the tank. When the air-lift generated flow reaches the surface, the flow will be forced towards the side walls 20 of the tank, and from there the flow will naturally occur along the internal side walls and downwardly in the tank. The circulation is further driven by gravitational forces, that pull culture in the tank downwardly along the side walls and floor 30 of the tank, towards the trough 35 in the floor 30, where circulation is completed by the airlift provided by the gas inlets 40,45,46. A critical feature of the airlift is that it is driven from the lowermost portion of the floor in the tank. This is to ensure that culture that settles at the bottom of the tank is lifted from the bottom, and thereby there is constant recirculation of culture in the tank, with minimal or no dead space in the tank (dead space meaning space through which there is little or no circulation of culture).

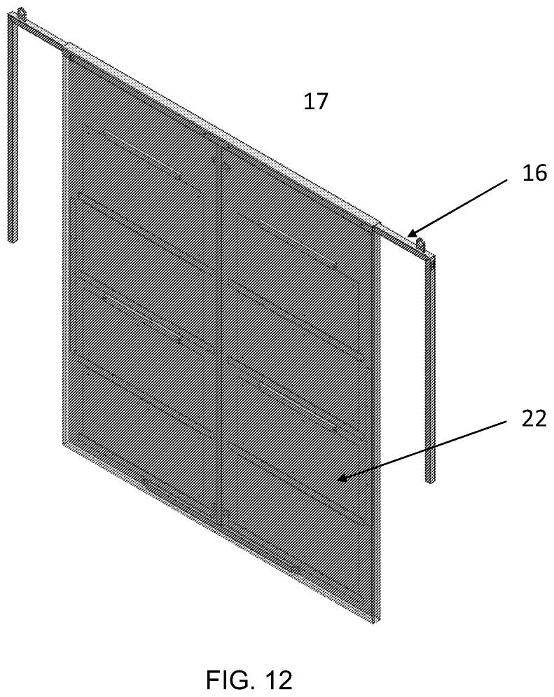

In FIG. 12, there is shown a closeup view of a rectangular illumination panel 17 in accordance with the invention. The illumination panel 17 contains a total of eight LED panels 22, arranged side by side within the panel so as to be in approximately the same plane. The illumination panel contains support 16 that can be used to suspend the panel from a frame structure within the photobioreactor (not shown).

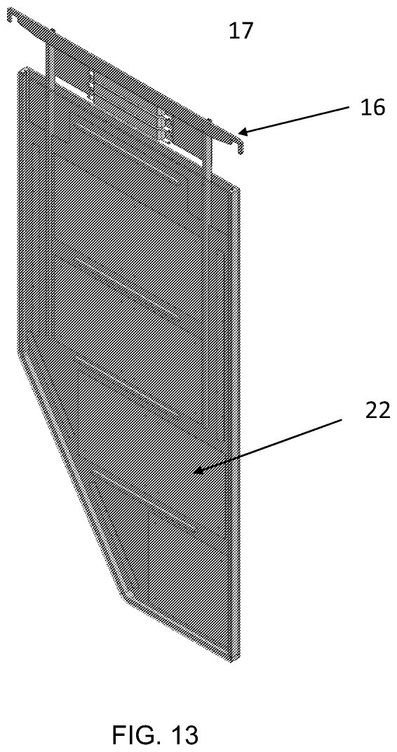

An alternative illumination panel 17 is illustrated in FIG. 13. Here, the panel contains a total of four LED panels 22. The panel has a pentagonal structure that can be suitable for use in a photobioreactor with curved side walls and/or floor panel, such as the photobioreactor shown in FIG. 2.

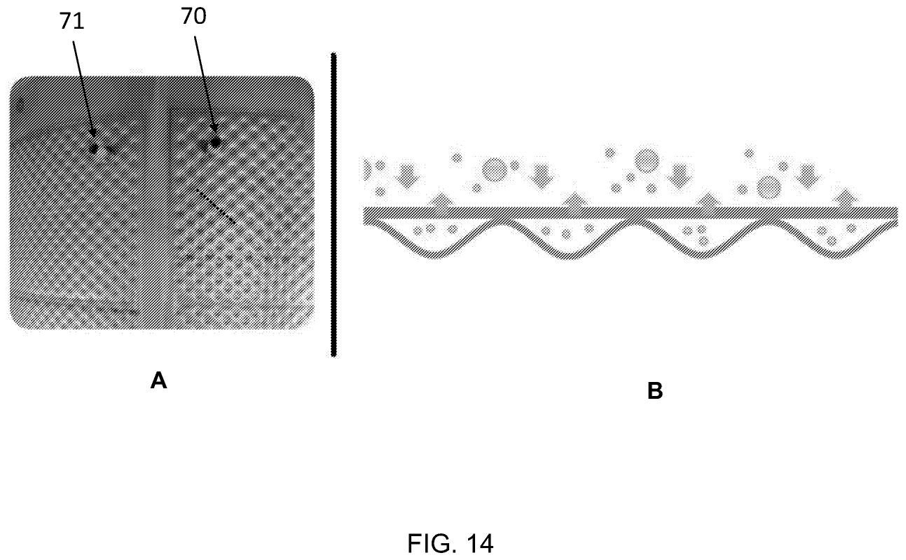

An embodiment of the thermal jacket is shown in FIG. 14. In (A), a side external (i.e., away from the interior of the tank, facing the outside) view of a thermal jacket is shown. The thermal jacket is provided by a continuous series of regularly arranged channels, interspersed by depressions that meet the underlying photobioreactor tank. In (B), a side view along the dotted line in (A) is shown. In this cross-sectional plane the thermal jacket forms a corregated structure, with free flow of liquid within each channel. In reality, the channels are interconnected, as is apparent from the side view in (A). The thermal jacket is provided in the outer surface (side panels and/or floor panel) of the photobioreactor tank, such that there is free flow of culture within the tank (upper half of figure in (B)) and a separate free flow of thermal fluid within the thermal jacket (lower half of (B)). Thus, the thermal jacket can be integral to the side and/or floor panels in the tank, as illustrated by the configuration shown in FIG. 14. The arrow across the two channels in (B) indicates thermal transfer across the photobioreactor tank wall, through which the temperature within the tank can be controlled by controlling the temperature of the thermal liquid circulating in the thermal jacket. Connectors 70,71 represent inlets and outlets, for delivering flow of thermal liquid (such as water) into or out from the thermal jacket. The temperature of the thermal liquid can be controlled by a separate temperature control unit (not shown), if so desired.

The skilled person will appreciate that the thermal jacket can be adapted in a variety of alternate manners consistent with the jacket allowing for circulation of fluid through the jacket, and the jacket being provided on the outer surface of the photobioreactor tank, so that there is thermal flow between tank and the thermal jacket.

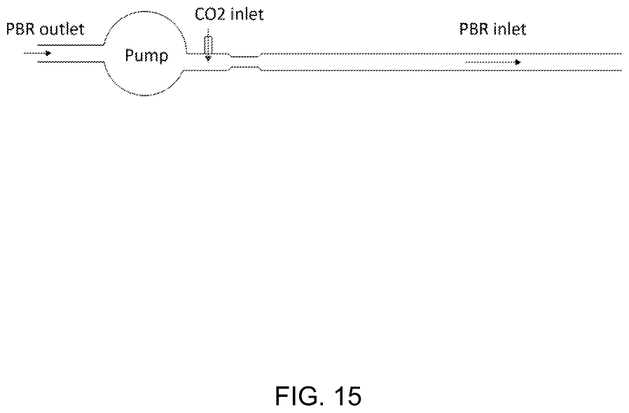

In FIG. 15, an embodiment showing delivery of CO₂ into the tank via a CO₂ injection loop in the photobioreactor is depicted. Culture from the tank is circulated through the CO₂ injection loope via a photobioreactor outlet (PBR outlet) by means of a circulation pump. Downstream of the pump there is a high-pressure zone due to the pumping of liquid against the mass of liquid in the tank. A CO₂ inlet is provided into this high-pressure zone, on which is provided a suitable CO₂ diffuser or sparger. The CO₂ injection loop finally connects with the photobioreactor tank via a PBR inlet, through which the culture, now rich in dissolved CO₂, reenters the tank. As a consequence of the delivery of CO₂ into a high-pressure environment, the dissolution of CO₂ in the water is maximized. As a consequence, there is a high conversion of CO₂ to biomass in the system.

The placement of the PBR inlets and outlets are in principle irrelevant, which means that the inlet and outlet can be placed anywhere within the tank. It may however be convenient to place the PBR outlet near one end of an elongated tank, and the PBR inlet near the opposite end of the tank. This way, the CO₂ injection (recirculation) loop can extend along the tank, from one end (or close to one end) to the other end (or close to the other end).

This configuration results in improved solvation of CO₂, and as a consequence, improved conversion of CO₂ to biomass in the reactor. Thus, by comparison with a normal (tank only) design, the recirculation loop can result in an increase in CO₂ to biomass conversion from about 0.14 to about 0.27.

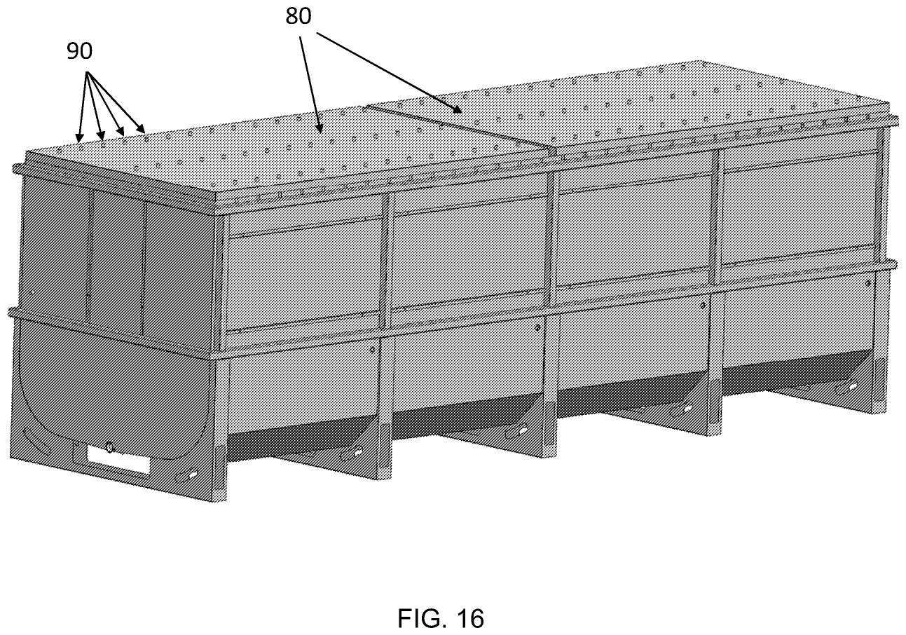

In FIG. 16, a closed photobioreactor in accordance with the invention is shown. The tank is closed in this embodiment by two lid units 80, each of which is essentially flat. On each lid units there are a number of sprinkler inlets 90. These inlets allow sprinkling solutions, such as detergent solutions or water to be sprinkled into the tank. Tubing and/or pipes (not shown) are connected to the sprinklers, and desired detergent and/or rinsing solutions pumped into the tank as desired. It should be appreciated that the lid units can alternatively be provided without sprinkler inlets, if so desired.

It is thus possible to clean the tank without opening the tank, by delivering cleaning solutions through the sprinkler inlets 90. Culture in the tank (e.g., algae culture) can be drained from the tank, the tank and its interior components including light panels subsequently rinsed with water and/or cleaned by means of one or more detergent or disinfectant solutions by delivering these solutions through sprinklers 90 in the lid. As a consequence, the system is a closed, self-contained system that can be kept closed at all times. This way, contamination is minimized, as is evaporation from the tank during growth. The culturing also becomes more economical, and the downtime of the tank is kept to a minimum.

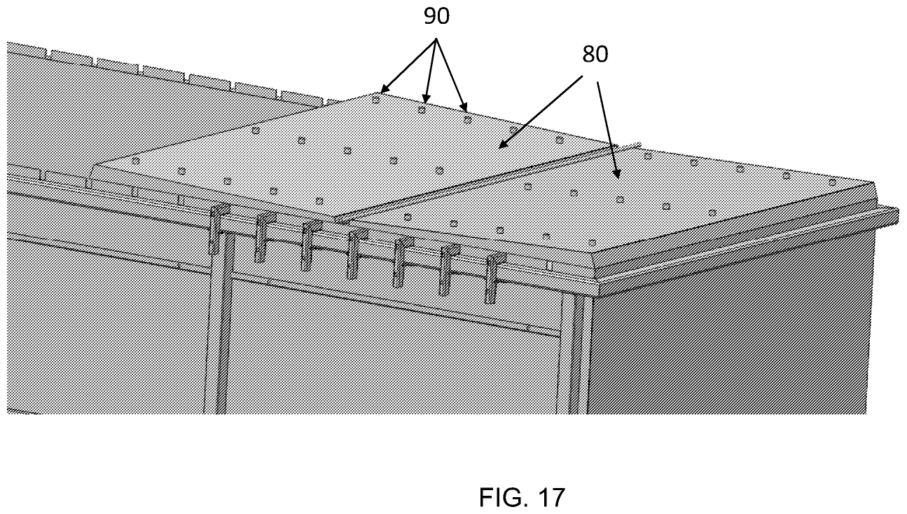

An alternative embodiments of a lid structure is shown in FIG. 17. In this embodiment, the tank can be closed by six lid units (only two are shown in this figure). Each of the units 80 has a sloping upper surface, so that any liquid that accumulates on the lid will flow off the lide. Sprinklers 90 are arranged on each of the lid units 80, for the delivery of washing and/or rinsing solutions.

There can be one or more supply tanks (not shown) from which detergent and/or rinsing solutions are pumped via the sprinklers 90 into the tank for cleaning purposes. Thus, the photobioreactor may comprise one or more detergent stock solution container, at least one pump, and tubes or piping for connecting to sprinklers installed in the lid of the photobioreactor, so as to ensure effective cleaning of the interior of the photobioreactor tank, including its interior light panels.

It will be appreciated that other configurations of the lid structure is possible and within scope of the invention. Thus, the tank can be closed off by a single lid unit if so desired. Alternate shapes and numbers of lid units that in combination cover the tank than shown here are also possible.

In summary, the circulation path within the tank can thus be described as being (i) vertical from the trough in the tank, towards the surface of liquid culture in the tank, (ii) horizontal, towards the internal walls of the tank, and (iii) vertical along the internal side walls in the tank, followed by a combination of vertical and horizontal movement towards the trough in the floor of the tank.

As will be appreciated from the foregoing description, the position of illumination in the tank that is parallel to the horizontal movement in this circulation path means that the illumination panels do not impede the circulation within the tank. Since the illumination panels also do not restrict flow along the side walls of the tank, there will also be unrestricted flow along the side walls and floor of the tank, towards its bottom (trough).

As will also be appreciated that the photobioreactor design described herein is geared to ensure stability and feasibility in the biomass production of e.g. microalgae. Ensuring stable production, free of contamination and other growth problems, has been a problem with prior art tank design. The photobioreactor described herein circumvents or prevents such problems, ensuring constant, secure cultivation of microalgae for the production of e.g. proteins and biomaterials such as asthaxanthin.

The present invention thereby provides a system and method for the growth of photosensitive organisms, driven by airlift that provides efficient circulation in the tank and at the same time adjustable and efficient illumination provided by the submerged illumination panels in the tank.

The present invention thus provides a number of advantages over prior art photobioreactors:
User-friendly, highly adaptable and industrial sized photobioreactors
High volumetric productivity
High aerial productivity—high culture volume per ground footprint
High biomass production per ground footprint compared with other cultivation methods
Safe harvesting and pest avoidance using an integrated photobioreactor
Internal panel illumination that can be regulated and adapted, resulting in high photosynthetic efficiency
Highly cost-effective production of biomass, due to high production per ground footprint
Environmentally friendly production, involving minimal use of CO₂, water and energy, resulting in high productivity of the tank

Throughout the description and claims, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

Embodiments of the present invention include:
1. A photobioreactor, in particular for culturing light sensitive microbes, the photobioreactor comprising
   a. a body comprising at least one floor panel and at least one wall extending upwardly around the periphery of the at least one floor panel to define a body for receiving and holding liquids; wherein the at least one floor panel comprises at least one graded segment so as to define a trough along the bottom of the body towards which debris within the body flows;
   b. at least one illumination panel, the at least one illumination panel comprising illumination means adapted to emit light that is suitable for sustaining growth of the light sensitive microbes within the photobioreactor,
   wherein the at least illumination panel is disposed within the body so that there is free flow of fluid along the sides and floor panel of the body;
   and
   c. at least one gas inlet disposed within the trough in the at least one floor panel, for providing an upward stream of gas into the body so as to generate air lift of the light sensitive microbes within the body;
   so that, during incubation of a liquid culture of microbes in the photobioreactor, microbe circulation and growth in the photobioreactor is stimulated through concomitant exposure to light emitted by the at least one illumination panel and circulation of microbes within the body that is facilitated by an upward air lift generated by the stream of gas into the body.
2. The photobioreactor of embodiment 1, wherein the at least illumination panel is disposed within the body so that there is at least one gap between the at least one panel and the at least one wall and/or the floor panel of the photobioreactor.
3. The photobioreactor of embodiment 1 or embodiment 2, wherein the illumination panel is provided in the form of at least one flat panel that is disposed within the body.
4. The photobioreactor of embodiment 3, wherein the angle between the illumination panel and the side walls is in the range of 45° to 90°, preferably in the range of 60° to 90°, more preferably in the range of 70° to 90°, more preferably in the range of 80° to 90°, more preferably in the range of 85° to 90°, even more preferably in the range of about 90°.
5. The photobioreactor of any one of the preceding embodiments, wherein the body comprises an elongate structure comprising at least two side walls and at least two end walls, and wherein the trough extends longitudinally along the floor panel of the body.
6. The photobioreactor of embodiment 5, wherein the trough extends longitudinally along the middle of the floor panel.
7. The photobioreactor of embodiment 5 or embodiment 6, wherein the side walls of the body have a rectangular structure.
8. The photobioreactor of any one of the preceding embodiments, wherein the floor panel comprises at least one downwardly convex segment.
9. The photobioreactor of any one of the preceding embodiments, wherein the floor panel comprises a continuously downwardly convex structure, so that debris can flow towards the lowermost part of the floor panel.
10. The photobioreactor of any one of the preceding embodiments, wherein the floor panel comprises a downwardly convex structure that merges into a flat strip at its lowermost point that stretches along the middle of the floor panel.
11. The photobioreactor of any one of the preceding embodiments, wherein the floor panel comprises a half-cylindrical structure having a semicircular cross-section, when viewed along the longitudinal axis of the body.
12. The photobioreactor of any one of the preceding embodiments, wherein the at least one gas inlet is disposed in the floor panel.
13. The photobioreactor of the preceding embodiment, wherein the at least one gas inlet comprises delivery spout that extends no more than 10mm into the body of the photobioreactor, preferably no more than 5 mm, even more preferably no more than 3 mm.
14. The photobioreactor of any one of the preceding embodiments, wherein the at least one gas inlet is disposed in the floor panel of the body, below the at least one illumination panel.
15. The photobioreactor of any one of the preceding embodiments, wherein the at least one gas inlet comprises a plurality of gas inlets that are provided in the lowermost section of the floor panel.
16. The photobioreactor of any one of the preceding embodiments, wherein the at least one gas inlet comprises a plurality of gas inlets that are provided along the trough in the floor panel.
17. The photobioreactor of any one of the preceding embodiments, wherein the at least one gas inlet comprises a plurality of gas inlets that are intermittently arranged along the trough in the floor panel.
18. The photobioreactor of any one of the preceding embodiments, wherein the at least one gas inlet comprises a plurality of gas inlets that are arranged at regular intervals along the trough in the floor panel.
19. The photobioreactor of any one of the preceding embodiments, wherein the at least one illumination panel comprises a parallel arrangement of a plurality of illumination panels and wherein a plurality of gas inlets are arranged in the floor panel, below each of the illumination panels.
20. The photobioreactor of any one of the preceding embodiments, wherein the at least one illumination panel comprises a parallel arrangement of a plurality of illumination panels and wherein a plurality of gas inlets are arranged in the floor panel, between the illumination panels.
21. The photobioreactor of any one of the preceding embodiments, wherein the floor panel comprises a continuous convex structure of generally half-cylindrical shape, and wherein the at least one gas inlet comprises a first row of gas inlets that is arranged along the bottom of the half-cylindrical surface, and at least one further row of gas inlets that is arranged in the photobioreactor floor panel along its longitudinal axis, upwardly from and parallel to the first row of gas inlets.
22. The photobioreactor of any one of the preceding embodiments, wherein the gas inlets have an internal diameter in the range of about 1-10 mm, preferably in the range of about 2-8 mm, more preferably in the range of about 2-6 mm, more preferably in the range of about 3-5 mm.
23. The photobioreactor of any one of the preceding embodiments, wherein the illumination means is embedded within the illumination panel, so that a water-tight seal separates the illumination means from their surroundings.
24. The photobioreactor of any one of the preceding embodiments, wherein the illumination panel comprises illumination means that provide illumination away from both sides of the panel along their entire surface.
25. The photobioreactor of embodiment 16, wherein the illumination panel comprises a plurality of light sources that provide approximately uniform illumination away from both sides of the illumination panel along substantially its entire surface.
26. The photobioreactor of any one of the preceding embodiments, wherein the illumination panel comprises a plurality of light sources that are disposed so as to provide illumination away from both sides of the illumination panel, into the body of the photobioreactor.
27. The photobioreactor of any one of the preceding embodiments, wherein the illumination means is provided as a plurality of LED light sources that are embedded in a light-transparent material, preferably light-transparent plastic material.
28. The photobioreactor of any one of the embodiments 23 to 27, wherein the body comprises a generally elongate structure, and wherein the illumination panel provides illumination along the longitudinal axis of the body.
29. The photobioreactor of any one of the preceding embodiments, wherein the illumination panels comprise illumination means that are adapted to deliver light with a photon flux in the range of 20-1200 μmol/m²s, preferably 50-800 μmol/m²s, more preferably- 120-500 μmol/m²s.
30. The photobioreactor of any one of the preceding embodiments, wherein the illumination panel comprises a rectangular structure of approximately uniform thickness that is in the range of about 0.5% to about 5% of the panel width.
31. The photobioreactor of any one of the preceding embodiments, wherein the illumination panel comprises a rectangular structure of approximately uniform thickness that is in the range of about 0.5 cm to about 5 cm.
32. The photobioreactor of any one of the preceding embodiments, wherein the illumination panel comprises material that transmits visible light.
33. The photobioreactor of any one of the preceding embodiments, wherein the illumination panel has a width that is in the range of about 80% to about 99% of the width of the body of the photobioreactor.
34. The photobioreactor of any one of the preceding embodiments, wherein the illumination panel has a height that is in the range of about 80% to about 99% of the body height from its lowermost point in the floor panel.
35. The photobioreactor of any one of the preceding embodiments, wherein the illumination panels are disposed within the body so that there is a gap between the illumination panels and the side walls and floor panel of the body that is in the range of about 2-20 cm, preferably in the range of about 5-15 cm.
36. The photobioreactor of any one of the preceding embodiments, wherein the illumination panel comprises a water-proof housing comprising light-transmitting material, and one or more light board that is disposed within the housing.
37. The photobioreactor of the preceding embodiment, wherein the one or more light comprises a plurality of rectangular, linear and/or linear light boards.
38. The photobioreactor of any one of the embodiments 1-35, wherein the illumination panel consists of a water-proof light board comprising a plurality of light sources.
39. The photobioreactor of any one of the preceding embodiments, wherein the gas inlets are adapted to provide a combined volume of gas into the body of the photobioreactor per minute that is in the range of 10% to about 30% of the volume of the body.
40. The photobioreactor of any one of the preceding embodiments, wherein the upright side walls and the upright end walls are approximately vertical.
41. The photobioreactor of any one of the preceding embodiments, wherein the body of the photobioreactor has a rectangular shape when viewed from above, and wherein the ratio of the length of the side walls to the width of end walls is in the range of about 1:1 to about 5:1.
42. The photobioreactor of any one of the preceding embodiments, wherein the body of the photobioreactor has a ratio of width, at its widest point, to height, that is in the range of about 0.5:1 to about 2:1, preferably 0.8:1 to about 1.5:1, more preferably about 1:1.
43. The photobioreactor of any one of the preceding embodiments, wherein the body has an internal volume that is in the range of about 200 L to about 40,000 L.
44. The photobioreactor of any one of the preceding embodiments, wherein the side walls have a length in the range of about 1-10 m, preferably in the range of about 2-8 m, more preferably in the range of about 3-7 m.
45. The photobioreactor of any one of the preceding embodiments, wherein the end walls have a length in the range of about 0.5-3 m, preferably in the range of about 1-3 m, more preferably in the range of about 2-3 m.
46. The photobioreactor of any one of the preceding embodiments, further comprising at least one water inlet, for delivering water into the photobioreactor, and at least one water outlet, for draining water from the photobioreactor.
47. The photobioreactor of any one of the preceding embodiments, further comprising a thermal jacket, wherein the thermal jacket comprises a hollow structure that encapsules at least a portion of the body.
48. The photobioreactor of the preceding embodiment, wherein the thermal jacket comprises at least one inlet and at least one outlet, for introducing and releasing water from the hollow structure, respectively.
49. The photobioreactor of any one of the preceding embodiments 5 to 43, wherein the body is enclosed within a frame structure comprising outer side walls that are disposed outwardly from at least the side walls of the body so as to enclose the body.
50. The photobioreactor of any one of the preceding embodiments, the photobioreactor further comprising at least one support member that extends downwardly from the floor panel and/or end walls of the body so as to provide support to the photobioreactor when placed on a flat surface.
51. The photobioreactor of any one of the preceding embodiments, the photobioreactor further comprising at least one of:
   a. one or more pH meter to provide a measure of pH of liquid in the body;
   b. one or more gas inlet for delivering a stream of carbon dioxide enriched gas into the body;
   c. one or more temperature probe;
   d. one or more chemical probe for detecting inorganic matter such as nitrate; and
   e. one or more optical density and/or light density meter.
52. The photobioreactor of any one of the preceding embodiments, further comprising at least one controller, for controlling at least one of: illumination, water temperature, pH and air flow in the photobioreactor.
53. The photobioreactor of the preceding embodiment, wherein the controller is adapted to adjust at least one or more parameter selected from light intensity, temperature and pH, in response to measurements of microbe density within the body, as determined by optical density and/or light density measurements.
54. A method of growing light sensitive microbes, the method comprising:
   a. providing a photobioreactor, the photobioreactor comprising
      i. a body comprising a floor panel and at least one wall extending upwardly around the periphery of the floor panel to define a body for receiving and holding an aqueous solution comprising microbes; wherein the floor panel comprises at least one graded segment so as to define a trough along the bottom of the body towards which the microbes can flow;
      ii. at least one illumination panel that is disposed within the body, the illumination panel comprising illumination means adapted to emit light that is suitable for sustaining growth of the light sensitive microbes within the body,
      wherein the at least one illumination panel is disposed within the body so that there is free flow of water along the at least one wall and/or floor panel; and
      iii. at least one gas inlet disposed in the trough in the floor panel, for providing an upward stream of gas in the body so as to generate air lift of the light sensitive microbes within the body;
   b. providing an aqueous culture of light-sensitive microbes into the body so that the illumination panels are at least partially submerged;
   c. delivering gas into the aqueous culture in the body through the at least one gas inlet, thereby generating air-lift in the body that provides an upwardly flow of microbes from the at least gas inlet and a downwardly gravitational flow of microbes at least along and down the at least one side wall, towards the trough in the floor,
   d. providing illumination in the body by means of the at least one illumination panel;
   whereby microbe growth is stimulated through the airlift-driven circulation of the microbes within the body and exposure to light emitted by the at least one illumination panel.
55. The method of the previous embodiment, wherein gas is delivered into the aqueous culture through the at least one gas inlet at a pressure that is in the range of about 0.1-0.5 bar, preferably about 0.2-0.4 bar.
56. The method of any one of the previous embodiments 54 or 550, wherein gas is delivered into the photobioreactor through the at least one gas inlet at a combined total flow rate into the body per minute that is in the range of 10% to about 30% of the volume of the body.
57. The method of any one of the previous embodiments 54-56, wherein the photobioreactor comprises at least pH probe and/or at least one temperature probe, and wherein during growth, at least one of pH, temperature, illumination, inorganic matter such as nitrides, optical and/or ligh density and gas flow is selectively controlled.
58. The method of embodiment 57, wherein the controlling is achieved by at least one electronic controller.
59. The method of any one of the previous embodiments 54-58, wherein the gas is ambient air, optionally mixed with carbon dioxide.
60. The method of any one of the previous embodiments 54-59, wherein the gas is delivered into the tank at a total volume per volume of culture in the tank, that is in the range of about 50 to about 500 L/m³/min, preferably about 100 to about 400 L/m³/min, more preferably in the range of about 200 to about 300 L/m³/min.
61. The method of any one of the previous embodiments 54-60, wherein the body further comprises at least one inlet for delivering carbon dioxide gas into the body, and wherein during growth, pH of the microbe culture is controlled by adjusting the rate of carbon dioxide gas flow through the at least inlet into the body.
62. The method of any one of the previous embodiments 54-61, further comprising delivering at least one source of nutrients into the body for sustaining growth of the microbes in the photobioreactor.
63. The method of any one of the previous embodiments 54-62, wherein gas is delivered into the body of the photobioreactor so as to generate a vertical airlift from the trough in the floor of the body, and simultaneously allowing down-flow of microbes along and down the side walls and the floor, towards the trough in the floor.
64. The method of any one of the previous embodiments 54-63, wherein the method is performed using a photobioreactor as set forth in any one of the embodiments 1-53.

The invention will now be illustrated by the following non-limiting examples.

### EXAMPLE 1

### Cultivation of Haematococcus pluvialis

For the autotrophic production of biomass of the algae Haematococcus pluvialis for astaxanthin production in a photobioreactor (e.g. a photobioreactor as described herein) in a batch cultivation mode, the culturing is performed over 14 days, 7 days of green vegetative growth followed by 7 days of a astaxanthin formation period.

The vegetative growth is characterized by green cell-growth in size and active cell division and multiplication. The red phase is a process of cell encystment, characterized by plasmatic accumulation of astaxanthin and build-up of thick and sturdy for cell-wall for protection and survival of the cell.

Prior to cultivation the internal surfaces of the tank and light panels are cleaned. The tank is subsequently filled with water, the water is chlorinated as needed and heated typically from 7° C. to 25° C. by heating up the tank jacket for temperature regulation overnight. Then the water is dechlorinated by means of sodium thiosulphate and active aeration by injection of air through air-sparges located in the bottom of the tank. The aeration is continued for air-lift purpose, to keep the algae in suspension.

Inoculum of H. pluvialis is produced through step-up culturing in flasks from multiple 1 liter flasks to multiple 5 liter flasks under aseptic conditions using Kuhl-medium (Kuhl, A. & Lorenzen, H. (1964) Handling and culturing of Chlorella. In: D. M. Prescott, ed., Methods in cell physiology. Vol. 1, pp. 152-187, Academic Press, New York and London, 1964). Five liters flask cultures are then applied to inoculate a 1000 L inoculum photobioreactor which is propagated until the cell density has reached optical density around 1.5 absorbance units at 750 nm.

A 1000 L inoculum photobioreactor (PBR) is filled up with water and nutrients added in the form of Kristalon, a all-in-one formula in an appropriate concentration. After the water has reached 25° C. the flask inoculum cultures are added to the tank making up an optical density of approximately 0.3 absorbance units at 750 nm. The PBR inoculation culture is propagated until the cell density has reached approximately 1.5-2.5 absorbance units at 750 nm. Then the inoculation culture from the inoculation tank is transferred to production PBR which has at that point been filled up to around 80% of the tank volume with tap water, heated up to 25° C. and amended with Kristalon nutrient blend. The production tank is subsequently filled up with water. The production culture is then monitored in terms of growth of the alga, nutrient depletion and morphological changes of the algal cells. Depletion of potassium nitrate as the principal nutrient component is important to enhance astaxanthin formation. At the end of period of vegetative growth the biomass level has reached typically 3-3.5 g/L.

As the green vegetative growth phase is terminated the red phase starts; astaxanthin induction is triggered by creating a stress effect upon the alga which reacts by producing astaxanthin as a stress relief, principally comprising quenching of oxidative stress. A high intensity illumination is an effective stress factor. At the beginning of the red phase the illumination level is raised from approximately 250 to 700 μmol/m²/s by turning on extra light units mounted on the light panels. In addition, the algae is also stressed by raising the salinity through adding sodium chloride typically creating salinity of 0.8-1.0%. After 7 days of red phase the cell mass contains typically 3.5-4% astaxanthin on a dry weight basis. Eventually the biomass is harvested and processed for astaxanthin down-streaming.

### EXAMPLE 2

### Cultivation of Marine Algae

Marine algae can in general be cultivated using the photobioreactors in accordance with the present invention. Culturing can in general terms follow the culturing described in the above under Example 1.

In the case of culturing marine microalgae such as Nannochloropsis oculata in the tank for oil production, in a similar way the up-scaling of inoculum from flask cultures involves step-up culturing of the alga in liters of seawater or seawater equivalent to tenths of liters under aseptic conditions supplied with an appropriate nutrient solution such as “f/2 medium” (Andersen, R. A., Morton, S. L., and Sexton, J. P. 1997. Provasoli-Guillard National Center for Culture of Marine Phytoplankton 1997 list of strains. J. Phycol. 33 (suppl.):1-75). The flask cultures are transferred to an inoculum PBR and cultivated for about one week until the desired cell density is achieved. The inoculum is then transferred to the production tank managed in a similar way as described above in case of Haematococcus pluvialis. When the vegetative growth phase is terminated, principal nutrient elements should be depleted which will trigger the synthesis of cytoplasmic oil.

1. A photobioreactor, in particular for culturing light sensitive microbes, the photobioreactor comprising
a. a body comprising at least one floor panel and at least one wall extending upwardly around the periphery of the at least one floor panel to define a body for receiving and holding liquids; wherein the at least one floor panel comprises at least one graded segment so as to define a trough along the bottom of the body towards which debris within the body flows;
b. at least one illumination panel, the at least one illumination panel comprising illumination means adapted to emit light that is suitable for sustaining growth of the light sensitive microbes within the photobioreactor,
wherein the at least illumination panel is disposed within the body so that there is free flow of fluid along the sides and floor panel of the body;
and
c. at least one gas inlet disposed in the at least one floor panel, within the trough in the at least one floor panel, for providing an upward stream of gas into the body so as to generate air lift of the light sensitive microbes within the body;
so that, during incubation of a liquid culture of microbes in the photobioreactor, microbe circulation and growth in the photobioreactor is stimulated through concomitant exposure to light emitted by the at least one illumination panel and circulation of microbes within the body that is facilitated by an upward air lift generated by the stream of gas into the body.

2. The photobioreactor of claim 1, wherein the at least illumination panel is disposed within the body so that there is at least one gap between the at least one panel and the at least one wall and/or the floor panel of the photobioreactor.

3. The photobioreactor of claim 1, wherein the illumination panel is provided in the form of at least one flat panel that is disposed within the body.

4. The photobioreactor of claim 3, wherein the angle between the illumination panel and the side walls is in the range of 45° to 90°.

5. The photobioreactor of claim 1, wherein the body comprises an elongate structure comprising at least two side walls and at least two end walls, and wherein the trough extends longitudinally along the floor panel of the body.

6. The photobioreactor of claim 5, wherein the trough extends longitudinally along the middle of the floor panel.

7. (canceled)

8. (canceled)

9. The photobioreactor of claim 1, wherein the floor panel comprises a continuously downwardly convex structure, so that debris can flow towards the lowermost part of the floor panel.

10. (canceled)

11. (canceled)

12. The photobioreactor of claim 1, wherein the at least one gas inlet is disposed in the floor panel.

13. The photobioreactor of claim 12, wherein the at least one gas inlet comprises delivery spout that extends no more than 10 mm into the body of the photobioreactor.

14. The photobioreactor claim 1, wherein the at least one gas inlet is disposed in the floor panel of the body, below the at least one illumination panel.

15. The photobioreactor of claim 1, wherein the at least one gas inlet comprises a plurality of gas inlets that are provided in a lowermost section of the floor panel.

16. (canceled)

17. (canceled)

18. (canceled)

19. (canceled)

20. The photobioreactor of claim 1, wherein the at least one illumination panel comprises a parallel arrangement of a plurality of illumination panels and wherein a plurality of gas inlets are arranged in the floor panel, between the illumination panels.

21. The photobioreactor of claim 1, wherein the floor panel comprises a continuous convex structure of generally half-cylindrical shape, and wherein the at least one gas inlet comprises a first row of gas inlets that is arranged along the bottom of the half-cylindrical surface, and at least one further row of gas inlets that is arranged in the photobioreactor floor panel along its longitudinal axis, upwardly from and parallel to the first row of gas inlets.

22. (canceled)

23. The photobioreactor of claim 1, wherein the illumination means is embedded within the illumination panel, so that a water-tight seal separates the illumination means from their surroundings.

24. (canceled)

25. (canceled)

26. (canceled)

27. (canceled)

28. The photobioreactor of claim 23, wherein the body comprises a generally elongate structure, and wherein the illumination panel provides illumination along the longitudinal axis of the body.

29. The photobioreactor of claim 1, wherein the illumination panels comprise illumination means that are adapted to deliver light with a photon flux in the range of 20-1200 μmol/m²s.

30. (canceled)

31. (canceled)

32. (canceled)

33. (canceled)

34. The photobioreactor of claim 1, wherein the illumination panel has a height that is in the range of about 80% to about 99% of the body height from its lowermost point in the floor panel.

35. The photobioreactor of claim 1, wherein the illumination panels are disposed within the body so that there is a gap between the illumination panels and the side walls and floor panel of the body that is in the range of about 2-20 cm.

36. (canceled)

37. (canceled)

38. (canceled)

39. (canceled)

40. (canceled)

41. (canceled)

42. (canceled)

43. The photobioreactor of claim 1, wherein the body has an internal volume that is in the range of about 200 L to about 40,000 L.

44. The photobioreactor of claim 1, wherein the side walls have a length in the range of about 1-10 m.

45. The photobioreactor of claim 1, wherein the end walls have a length in the range of about 0.5-3 m.

46. The photobioreactor of claim 1, further comprising at least one water inlet, for delivering water into the photobioreactor, and at least one water outlet, for draining water from the photobioreactor.

47. The photobioreactor of claim 1, further comprising a thermal jacket, wherein the thermal jacket comprises a hollow structure that encapsules at least a portion of the body.

48. The photobioreactor of claim 47, wherein the thermal jacket comprises at least one inlet and at least one outlet, for introducing and releasing water from the hollow structure, respectively.

49. The photobioreactor of claim 5, wherein the body is enclosed within a frame structure comprising outer side walls that are disposed outwardly from at least the side walls of the body so as to enclose the body.

50. (canceled)

51. The photobioreactor of claim 1, further comprising at least one CO₂ recirculation loop that is fluidly connected to the body via at least one recirculation loop tank outlet and at least one recirculation loop tank inlet, and at least one pump, for pumping culture through the CO₂ recirculation loop, the CO₂ recirculation loop further comprising at least one CO₂ inlet that is provided downstream of the at least recirculation pump, with respect to the direction of liquid flow in the CO₂ recirculation loop.

52. The photobioreactor of claim 51, wherein the at least one recirculation loop tank outlet and the at least one recirculation loop tank inlet are provided in the lower half of the body, with respect to the volume of the body.

53. (canceled)

54. The photobioreactor of claim 1, the photobioreactor further comprising at least one of:
a. one or more pH meter to provide a measure of pH of liquid in the body;
b. one or more gas inlet for delivering a stream of carbon dioxide enriched gas into the body;
c. one or more temperature probe;
d. one or more chemical probe for detecting inorganic matter such as nitrate; and
e. one or more optical density and/or light density meter.

55. The photobioreactor of claim 1, further comprising at least one controller, for controlling at least one of: illumination, water temperature, pH and air flow in the photobioreactor.

56. The photobioreactor of claim 55, wherein the controller is adapted to adjust at least one or more parameter selected from light intensity, temperature and pH, in response to measurements of microbe density within the body, as determined by optical density and/or light density measurements.

57. A method of growing light sensitive microbes, the method comprising:
a. providing a photobioreactor, the photobioreactor comprising
i. a body comprising a floor panel and at least one wall extending upwardly around the periphery of the floor panel to define a body for receiving and holding an aqueous solution comprising microbes; wherein the floor panel comprises at least one graded segment so as to define a trough along the bottom of the body towards which the microbes can flow;
ii. at least one illumination panel that is disposed within the body, the illumination panel comprising illumination means adapted to emit light that is suitable for sustaining growth of the light sensitive microbes within the body,
wherein the at least one illumination panel is disposed within the body so that there is free flow of water along the at least one wall and/or floor panel; and
iii. at least one gas inlet disposed in the trough in the floor panel, for providing an upward stream of gas in the body so as to generate air lift of the light sensitive microbes within the body;
b. providing an aqueous culture of light-sensitive microbes into the body so that the illumination panels are at least partially submerged;
c. delivering gas into the aqueous culture in the body through the at least one gas inlet, thereby generating air-lift in the body that provides an upwardly flow of microbes from the at least gas inlet and a downwardly gravitational flow of microbes at least along and down the at least one side wall, towards the trough in the floor,
d. providing illumination in the body by means of the at least one illumination panel;
whereby microbe growth is stimulated through the airlift-driven circulation of the microbes within the body and exposure to light emitted by the at least one illumination panel.

58. (canceled)

59. The method of claim 57, wherein gas is delivered into the photobioreactor through the at least one gas inlet at a combined total flow rate into the body per minute that is in the range of 10% to about 30% of the volume of the body.

60. (canceled)

61. (canceled)

62. (canceled)

63. (canceled)

64. (canceled)

65. The method of claim 57, further comprising delivering at least one source of nutrients into the body for sustaining growth of the microbes in the photobioreactor.

66. The method of claim 57, wherein gas is delivered into the body of the photobioreactor so as to generate a vertical airlift from the trough in the floor of the body, and simultaneously allowing down-flow of microbes along and down the side walls and the floor, towards the trough in the floor.

67. (canceled)
